(19) United States
(12) Patent Application Publication
(10) Pub. No.: US 2003/0162741 A1
(43) Pub. Date: Aug. 28, 2003

(54) Oral DNA composition for hepatitis B virus chronic infection

(75) Inventors: Mun-Hon Ng, Bagulo Villa (HK); Kwok-Yung Yuen, Baguio Villa (HK); Bo-Jian Zheng, Pokfulam Garden (HK)

Correspondence Address: Sughrue Mion, PLLC, Suite 800, 2100 Pennsylvania Ave., N.W., Washington, DC, 20037-3213, US

(73) Assignee: THE UNIVERSITY OF HONG KONG

(21) Appl. No.: 10/375,143

(22) Filed: Feb. 28, 2003

Related U.S. Application Data
(60) Provisional application No. 60/359,945, filed on Feb. 28, 2002.

(51) Int. Cl. A61K 48/00

(52) U.S. Cl. 514/44; 424/93.2

(57) ABSTRACT

The present invention provides an oral DNA composition for improving an impaired immunity associated with chronic infection of hepatitis B virus (HBV) and for suppressing transgene expression for a protrated period of time comprising an attenuated strain of bacterial cells which preferentially target phagocytic cells of the intestinal mucosa, and which serve as a vehicle for a plasmid vector carrying one or more genes or complementary DNA coding for at least a portion of a hepatitis B viral protein or peptide. Given orally, the DNA composition causes a transient and self-limiting infection of the intestinal tract through autolysis of the bacterial cells and release of the plasmid after gaining entry into infected host cells. A promotor contained within the plasmid allows for expression of the HBV gene(s) in the eurokaryotic environment, the viral products of which help to booster a cell-mediated immunity to clear the infection and reverse a state of immune tolerance characteristic of HBV chronic infection.

### FIELD OF THE INVENTION

The present invention relates to an oral DNA composition (ODV) for ameliorating an impaired immunity in individuals who are chronically infected with hepatitis B virus (HBV). The oral DNA composition serves to booster immunity against HBV, improve the immune deficits associated with the disease and clear the infection.

### BACKGROUND OF THE INVENTION

The World Health Organization (WHO) estimated that there are 350 million people world wide, who are chronically infected with the hepatitis B virus (HBV) [1]. These individuals have a high risk of developing liver cirrhosis and liver cancer. In addition, being the only significant reservoir for HBV, these individuals also pose as a significant public health hazard. None of the treatments presently available for chronic HBV infection can clear the virus from these individuals and are only moderately effective in reducing virus replication [2-5].

The idea that HBV infection may be cleared through immune intervention is based on findings that acute self-limited HBV infection evokes vigorous, polyclonal T helper cell (Th) and cytotoxic T lymphocyte (CTL) responses against viral capsid and envelope antigens, leading to the clearance of the virus from the body. On the other hand, chronic HBV infection is associated with weak Th responses of a restricted spectrum of antiviral specificity and usually undetectable virus-specific CTL activity [6]. These findings suggested that an intact cell mediated immunity is the chief determinant of virus clearance and provided the rational basis for immune intervention of chronic HBV infection with the view to booster cell mediated immunity (CMI) against the virus in order to clear the infection [7]. The contention was further supported by findings from bone marrow transplantation showing that adoptive transfer of bone marrow cells from donors, who had acquired intact immunity against the virus from natural infection, can improve the immune deficits of the chronically infected recipients and thereby clear the infection [8].

Based on the above, candidate vaccines, or other means of immune intervention, for the treatment of chronic hepatitis are selected initially for their capacity to evoke a vigorous CMI in mice and they are further assessed in transgenic mice harboring part or a whole of the HBV genome. Expression of the viral transgene during the embryonic stage apparently had induced a state of immune tolerance in these animals, which is similar to that condition which prevails in chronically infected humans [9]. Since there is no animal that can be chronically infected with HBV, these animals are commonly used as a convenient model to assess efficacy of experimental vaccines for the treatment of chronic HBV infection [9-11]. Those experimental vaccines having the capacity to (1) evoke a vigorous CMI in immune competent mice, (2) reverse the state of immune tolerance, and (3) suppress transgene expression in the HBV transgenic animals, are considered to be potential candidate vaccines for immune intervention of chronic hepatitis B infection in humans.

Current HBV vaccines are protein vaccines, made up of recombinant HBV surface antigen (HbsAg). They generally evoke a vigorous antibody response and are effective in preventing the infection, but they do not evoke a vigorous CMI response considered to be suitable for the treatment of chronic infection. The capacity of protein vaccines to evoke a CMI response was enhanced by mixing the recombinant HBV vaccine with an optimum quantity of antibody [12]. The resulting immune complex vaccine evokes a more vigorous CMI response in immune competent mice than the parent recombinant vaccine and it also breaks the state of immune tolerance prevailing in transgenic mice [13]. However, the level of immunity induced by the immune complex vaccine was not sufficient to additionally suppress transgene expression.

An alternate approach has been to develop DNA vaccines for treatment of chronic HBV infection. The DNA vaccines evoke a more vigorous CMI than the protein vaccines in immune competent mice and they possess the capacity to break immune tolerance prevailing in HBV transgenic mice, but generally they too are incapable of suppressing transgene expression [10, 13-16]. The only known exception was one study described by Mancini et al. [17] however, it could not be ascertained whether the suppression observed in this study was induced by vaccination or whether it occurred spontaneously in the particular strain of transgenic mice used in their study. As best as can be determined, the only instance when suppression of transgene expression was indeed induced by vaccination was one which made use of a combination of the immune complex vaccine and DNA vaccine through repeated administration of both vaccines [13].

### SUMMARY OF THE INVENTION

It is an object of the present invention to provide an oral DNA composition for improving an impaired immunity associated with chronic infection of hepatitis B virus (HBV) and for suppressing transgene expression for a protracted period of time. According to the present invention, there is provided an oral DNA composition for improving an impaired immunity associated with chronic infection of HBV and for suppressing transgene expression for a protracted period of time comprising:

an attenuated strain of bacteria which preferentially targets phagocytic cells, wherein cells of the bacterial strain are transformed by a plasmid vector comprising:

one or more genes, or complementary DNA thereof, coding for at least a portion of a hepatitis B viral protein or peptide or antigenic portion thereof;

a promoter operably linked to the gene or complementary DNA permitting expression thereof in an eukaryotic environment; and

an auxotrophic mutation which causes the cells of the bacterial strain to undergo autolysis once they have gained entry into the phagocytic cells; and

a pharmaceutically acceptable carrier.

According to another aspect of the present invention, there is provided a process for inducing a cell-mediated immune response in a chronically infected HBV carrier comprising:

orally administering to the HBV carrier an effective amount of an attenuated bacterial strain which preferentially targets phagocytic cells of the intestinal mucosa, wherein cells of the bacterial strain undergo autolysis when taken up by the phagocytic cells, thereby causing release of a plasmid vector contained therein which is capable of expressing at least a portion of a HBV genome in an eukaryotic environment; and

inducing a cell-mediated immune response in the HBV carrier and suppressing HBV esxpression.

### BRIEF DESCRIPTION OF THE DRAWINGS

The invention will now be described in greater detail with reference to the drawings in which:

FIG. 1 is a schematic representation of the structure of a HBsAg-expressing plasmid pRc/CMV-HBs (S) comprising the plasmid vector, pRc, that harbors the CMV promotor linked to the vaccine gene (HBs (S)) that encodes the hepatitis B virus (ayw) surface antigen.

FIG. 2 illustrates a Lymphocyte proliferation assay in which three mice immunized intramuscularly with 3 doses of DNA composition every 3 weeks and the animals were sacrificed on week 8. Splenocytes harvested from the immunized animals were seeded in triplicate microtiter plates cultures containing 5×10⁵/well. The cultures were stimulated with peritoneal MF infected with ODV (M-ODV) or the carrier bacteria (M-BC); or MF that had been loaded with purified HBsAg (M-P). The immune splenocytes were also stimulated with irradiated P815-S, P815 or PHA respectively; or cultured without any stimulant as the control (Medium). The cultures were incubated for the indicated times and then labeled for a further 16 hours with [³H] thymidine. Lymphocyte proliferation indicated by mean cpm of [³H] thymidine incorporated by these cultures were compared with that obtained with control unstimulated cultures.

FIG. 3 illustrates an IFN-_{.}γ induction assay for the determination of HBsAg specific Th 1 cells. An ELISA was carried out to determine IFN-_{.}γ contents in supernatants collected at the indicated times from the same stimulated and unstimulated immune splenocyte cultures as described in FIG. 2.

FIG. 4 illustates a HBsAg specific Cytotoxic T cell precursor assay. The HBsAg immune splenocytes were co-cultured for 3 days with M-OVD, M-BC, M-P or irradiated P815-S at effector:stimulator ratio of 20. The cells were incubated further for 4 days in the presence of 25 IU/ml of murine rIL-2. Cytotoxicity against P815-S targets were determined for the stimulated immune splenocytes by a standard four-hour calcein release assay in triplicate at effector:target ratios between 30 and 0.3.

FIG. 5 illustrates serum anti-HBs responses to vaccination. Nine groups of five Balb C mice each were immunized with indicated immunogens. Serum samples were taken on the indicated times for determination of HBsAb as in (A). The samples taken on week 9 were further analyzed for contents of different subclass of the antibody as in (B).

FIG. 6 illustrates Th 1 and CTL responses to vaccination. Balb/C mice were vaccinated as in FIG. 5 and sacrificed on week 9 after vaccination. Splenocytes from the animals were cultured at 5×10⁶/ml in the presence of 10 mg/ml of purified protein HBsAg. The supernatants were taken at indicated times and measured for the secretion of IFN-_{.}γ_{.} by ELISA (A). The cultures were further incubated for an additional 4 to 5 days in the presence of 25 IU/ml of rIL-2. The cytotoxic activities in these cultures were determined by CTL assay (B).

FIG. 7 illustrates immunohistochemical staining of HBsAg expressed in liver sections from oral DNA vaccinated and bacterial carrier vaccinated HBs-Tg mice. Liver sections from HBs-Tg mice sacrificed 12 weeks after receipt of the oral DNA composition (A) or the bacterial carrier (B) were stained using the DAKO immunohistochemical kit to determine expression of the HBsAg transgene in hepatocytes (original magnification ×100). Note that all hepatocytes from the animals given bacterial carrier were positive for HBsAg (B, 4-A to -E) but the section from a normal B57/6J mouse was negative for the viral antigen (B, N—C). The liver tissues of oral DNA vaccinated mice (A, 3-A to -E) showed patchy expression of the viral antigen. There was a preponderance of cytolytic or necrotic HBsAg positive liver cells (<=) and HBsAg negative hepatocytes (<−) in the liver section from one oral DNA vaccinated mouse which died of fulminent hepatitis on day 13 post-vaccination (A, 3-F).

FIG. 8 illustrates histopathological analysis of liver sections from oral DNA vaccinated (A) and bacterial carrier vaccinated (B) HBs-Tg mice. Liver sections were prepared from the mice as described in the legend of FIG. 5 and stained with H&E (original magnification ×100). The section from the mouse that died of fulminent hepatitis on day 13 post-immunization exhibited intense lymphocytic inflammation with prominent eosinophic liver cell degeneration. Liver tissues from the other animals showed minimum or no pathology.

FIG. 9 illustrates oral DNA composition induced an early hepatitic flare in HBs-Tg mice. Liver sections were taken from oral DNA vaccinated mice (3) and their bacterial carrier controls (4) at the indicated weeks and stained by H&E (original magnification ×200). Intense focal inflammation accompanied by eosinophilic liver cell degeneration developed 2 weeks after receipt of the oral DNA composition (b & j). Inflammation subsided with scanty eosinophilic liver cell degeneration on week 3 (c), and minimum pathology was seen on week 4 (d). Mild focal inflammation with scanty eosinophilic hepatocyte degeneration was seen in bacterial carrier control animals 2 weeks after immunization (f) and subsequent liver samples showed minimum pathology (g & h).

FIG. 10 illustrates serum ALT levels in different groups of immunized HBs-Tg mice. Five groups of HBs-Tg mice were respectively immunized with indicated immunogens and the serum samples were collected at 3-week intervals (A). Serum samples were also obtained at 1-week intervals from two groups of 12 HBs-Tg mice each in the first 4 weeks after vaccinated with the oral DNA composition or the bacterial carrier (B). Average and SD values of serum ALT levels are shown for the indicated weeks after vaccination.

FIG. 11 illustrates oral DNA composition induced an early suppression of transgene in HBs-Tg mice. Liver sections were taken from oral DNA vaccinated mice (3) and their bacterial carrier controls (4) at the indicated weeks and examined for HBsAg expression by immunohistology (original magnification ×200). Immunohistology revealed a marked suppression of HBsAg expression at week 2 after received oral DNA vaccination (j), and substantial proportions of liver cells in positive liver cells (← or →) and apparently normal HBsAg negative hepatocytes (↑ or ↓) were found in these samples. While most liver cells from control animals were positive for HBsAg (m to p).

FIG. 12 illustrates serum anti-HBs levels and antibody subtypes in different groups of immunized HBs-Tg mice. Groups of HBs-Tg mice were respectively immunized with the indicated immunogens (A). Another set of HBs-Tg mice was immunized with either the oral DNA composition or the bacterial carrier (A′). Average and SD values of anti-HBs levels are shown for the indicated weeks post-vaccination. The subtypes of the antibodies in positive samples obtained at week 12 were presented as OD value ±SD (B).

FIG. 13 illustrates HBsAg levels (O.D.450) in the lysates of 293 cells transfected with pRc/CMV-HBs(S) harvested at 48 h post-transfection and macrophages infected with S. typhimurium pRc/CMV-HBs(S) harvested at 24, 48, and 74 h post-infection.

FIG. 14 illustrates serum antibody levels (O.D.492) at (A) day 7 and (B) day 21 in Balb/c mice immunized with intramuscular pRc/CMV-HBs(S), oral live-attenuated S. typhimurium, transformed with pRc/CMV-HBs(S), intraperitoneal recombinant HBsAg, and oral live-attenuated S. typhimurium.

FIG. 15 illustrates CTL response of Balb/c mice immunized with oral live-attenuated S. typhimurium, intramuscular pRc/CMV-HBs(S), oral live-attenuated S. typhimurium transformed with pRc/CMV-HBs(S), and intraperiotoneal recombinant HBsAg using P815 cells expressing HBsAg (P815S) and P815 cells not expressing HBsAg (P815N) as targets. Mice immunized orally with live-attenuated S. typhimurium transformed with pRc/CMV-HBs(S) showed significantly stronger CTL response than mice immunized intraperitoneally with recombinant HBsAg (p<0.01 at E:T ratio of 100:1), while comparable to mice immunized with intramuscular pRc/CMV-HBs(S) at all E:T ratios.

FIG. 16 illustrates (A) Interleukin-4 and (b) IFN-_{.}γ_{.} levels (O.D.450) of splenic cell culture supernatant at 24, 48, and 72 h in Balb/c mice immunized with intramuscular pRc/CMV-HBs(S), oral live-attenuated S. typhimurium transformed with pRc/CMV-HBs(S), intraperitoneal recombinant HBsAg, and oral live-attenuated S. typhimurium.

FIG. 17 illustrates serum HBsAg levels in different groups of immunized transgenic mice. Groups of mice were separately immunized intramuscularly with HBsAg 2 μg/mouse ( ), HBsAg-anti-HBs complex containing 2 μg HBsAg/mouse, abbreviated as IC ( ), IC containing 2 μg HBsAg combined with 100 μg of naked plasmid DNA with S gene/mouse, abbreviated as IC-sDNA ( ), 100 μg of naked plasmid DNA with S gene/mouse ( ) at 3-week intervals for four injections, and unimmunized control (x). Average and S.D. of serum HBsAg levels are presented as assayed on different weeks after immunization.

FIG. 18 illustrates serum anti-HBs antibody levels in different groups of immunized transgenic mice. Groups of mice were separately immunized intramuscularly as indicated in the description of FIG. 1. Average and S.D. of anti-HBs antibodies are presented as assayed on different weeks after immunization.

FIG. 19 illustrates a CTL response in different immunized groups. Groups of mice were separately immunized with HBsAg, IC, IC-sDNA or s-DNA. Mice were boosted 7 days prior to being sacrificed and T cells from mouse spleens were stimulated with HBsAg and further expanded by incubation with IL-2. Target cells used were splenocytes of normal C57/6J mice infected with Vac-HBsAg virus (A), while splenocytes infected with vaccinia virus (B) served as control. Percentages of specific cytolysis at effector cells/target cells (ranged from 100/1 to 170.3/1) are presented in HBsAg immunized group, IC immunized group, IC-sDNA immunized group, s-DNA immunized group and unimmunized group.

FIG. 20 illustrates an immunohistochemical staining of HBsAg expressed in liver section of IC-sDNA immunized and unimmunized control transgenic mice. (A) Liver sections of five transgenic mice which were immunized with IC-sDNA (as indicated in the description of FIG. 1) intramuscularly for four injections at 3-week intervals, sacrificed at week 15 and were stained for HBsAg by Dako immunohistochemical kit. In short, sections were first stained with goat anti-HBsAg overnight, followed by reacting with rabbit anit-goat biotinylated antibody for 30 min., washed and further reacted with streptavidin-HRP-conjugate for another 30 min. and finally, the substrate for horse radish peroxidase was added. Compared to the unimmunized control mice, in two out of the five immunized mice, HBsAg positive hepatocytes were observed. NC was a liver section from a normal control mouse. (B) Liver sections from six unimmunized control transgenic mice.

### DETAILED DESCRIPTION OF THE INVENTION

Unless defined otherwise, all technical and scientific terms used herein have the same meaning as commonly understood by one of ordinary skill in the art to which this invention belongs. Generally, the nomenclature used herein and the laboratory procedures in cell culture, molecular genetics, and nucleic acid chemistry described below are those well known and commonly employed in the art. Standard techniques are used for recombinant nucleic acid methods, cell culture and transformation. As employed throughout the disclosure, the following terms, unless otherwise indicated, shall be understood to have the following meanings:

Vaccine gene according to the present invention means one or more genes or complementary DNA coding for at least a portion of a hepatitis B protein or peptide, or an antigenic portion thereof.

Vaccine plasmid according to the present invention means a plasmid carrying one or more genes or complementary DNA coding for at least a portion of a hepatitis B protein or peptide, or an antigenic portion thereof, and which is capable of being expressed in an eukaryotic environment.

According to the present invention, there is provided an oral DNA composition, which in a single dose, can break HBV immune tolerance prevailing in transgenic mice and evoke a vigorous CMI response in these animals governed by long term suppression of transgene expression [18]. The oral DNA composition comprises two principle component parts: (1) an attenuated strain of bacteria; and (2) a plasmid vector that comprises one or more genes or complementary DNA coding for at least a portion of a hepatitis B protein or peptide, or an antigenic portion thereof, which is capable of transforming cells of the bacterial strain. Two features inherent to the oral DNA composition and its use thereof which render this composition more efficacious than presently known vaccines are: (1) the carrier bacteria which has the capacity to deliver the vaccine gene to professional antigen-presenting cells in the intestine; and (2) the plasmid vector which has the capacity to enable expression of the vaccine gene after it has been delivered to the cells of the intestine. Given orally, the oral DNA composition causes a transient and self-limiting infection of the intestinal tract. Efficacy of the oral composition is attributed to the following features: (1) the capacity of the carrier bacteria to deliver the vaccine plasmid preferentially to phagocytic cells, including those residing in the intestinal mucosa, and inflammatory cells recruited to the site in response to the infection; (2) a mutation that causes the bacteria to undergo autolysis after it has gained entry to the cells thereby releasing the vaccine plasmid into the infected cells; and (3) a CMV promotor contained within the vaccine plasmid that allows expression of the vaccine gene in an eukaryotic environment of the infected cells.Thus, unlike other vaccines, immunization using the oral DNA composition of the present invention essentially renders a process of infection in a similar manner to that which immunity is acquired in natural infection. Accordingly, it is the particular combination of the carrier bacteria and the plasmid vector which renders the oral DNA composition efficacious for treatment of chronic infection with hepatitis B virus. It is believed that the lack of one or both of these features makes other known vaccines less effective at reversing the state of immune tolerance when compared to the oral DNA composition of the present invention. It is expected that the combination of the same features would be important in development of vaccines for treatment of chronic infections caused by viruses other than HBV, such as hepatitis C virus (HCV) and human immunodeficiency virus (HIV).

The bacterial strains used are Salmonella typhimurium strain S7207 or Salmonella typhi strain Ty21a, both developed by Dr Stocker. The bacterial strain, Ty21a, is commercially available as a typhoid vaccine. The vaccine plasmid pRc/CMV-HBs(S) was developed by Dr Wheland as an experimental DNA vaccine for HBV infection. The oral DNA composition is formulated by transforming the bacteria with the vaccine plasmid by standard protocol. The combination of the two components has resulted in a new composition, which can be administered orally and which, in a single dose, can suppress HBV transgene expression in a strain of HBV transgenic mice.

The transgenic mice exhibited a state of immune tolerance to HBV that models the immune status of humans who are chronically infected with the virus. Because there is no animal model of chronic HBV infection, HBV transgenic mice are commonly used to assess experimental vaccines to determine if they are potentially suitable for treatment of chronic HBV infection in humans. As can be ascertained, no other vaccine developed to date has been shown to be capable of suppressing transgene expression in such model. The capacity of the oral DNA composition to effect long term transgene suppression makes it an effective composition for treating chronic HBV infection of humans and breaking the immune tolerance characteristic of the disease.

The oral DNA composition evoked a vigorous Th 1-type response in these animals and the development was temporally related to onset of suppression of transgene expression in liver tissue. This suggested that suppression was primarily brought about by immune, rather than innate, mechanisms. The contention is supported in control animals given the carrier bacteria. The bacteria also activated innate mechanisms, however it did not effect suppression of transgene expression.

Immune response evoked by other vaccines, especially the DNA vaccine, that failed to suppress transgene expression differs only quantitatively from that evoked by the oral DNA composition. This suggests that the vigorous Th1 type immune response evoked by oral composition is an important determinant of its efficacy for treatment of chronic infection with hepatitis B virus.

As can be ascertained, the oral composition is the first of its kind discovered that can effect suppression of transgene expression for protracted period of time. Hence, our claims pertain to formulation of oral DNA composition for the treatment of chronic infection with hepatitis B virus, specifically, and the same or similar vaccine formulation for the treatment of chronic infections with other viruses generally.

The following examples are provided to describe in detail some of the representative, presently preferred methods and materials of the invention. These examples are provided for purposes of illustration of the inventive concepts, and are not intended to limit the scope of the invention as defined by the appended claims.

### Example 1

### Component Parts, Formulation and Mode of Operation of Oral DNA Composition for Treatment of Chronic HBV Infection (ODV)

Component Parts

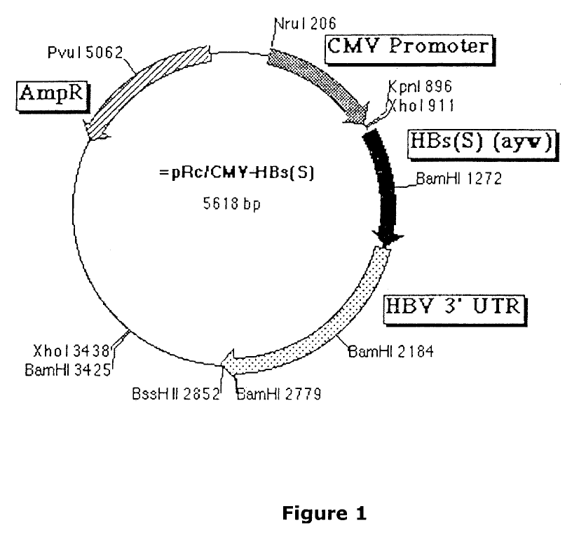

The oral DNA composition is made up of two component parts; these are a vaccine plasmid contained in a strain of carrier bacteria. The vaccine plasmid, pRc/CMV-HBs(S), was developed by Dr J Wehland [17,20]. As depicted in FIG. 1, it comprises the plasmid vector (pRc) that harbors a CMV promotor linked to the vaccine gene encoding HBsAg (S). This and such similar vaccine plasmid constructs provide for the vaccine gene to be amplified in suitable bacterial hosts and for the viral antigen it encodes to be expressed in eukaryotic cellular environment.

The carrier bacteria used was an attenuated strain of Salmonella typhi (S. ty 21a), a strain used to produce the commercial oral typhoid vaccine, or an attenuated strain of Salmonella typhimurium [S. typhimurium 2337-65 derivative hisG46, DEL 407 [aroA:Tn10{Tc-s}], referred hitherto as Salmonella typhimurium aroA strain SL 7207. Both strains, developed by Dr B Stoker [19], are established intracellular parasites adept to infect the intestinal tract, whereby the bacteria is taken up preferentially by lymphoid cells present in the intestinal mucosa, including APC. Both strains had been attenuated, carrying auxotrophic mutations that causes them to undergo autolysis once they have gained entry into host cells.

Formulation and Mode of Operation

The oral DNA composition (ODV) was produced by transforming the carrier bacteria with the HBV vaccine plasmid according to standard protocol. The composition is given orally or through incubation. The intestinal infection ensuing is transient and self-limiting because of the auxotropic mutations. The infection nevertheless serves to deliver the vaccine plasmid into intestinal cells, especially lymphoid cells, which include those that are already present in the intestinal mucosa, and inflammatory cells recruited to the site in response to the infection. The vaccine plasmid is released inside the infected cells upon autolysis of the carrier bacteria and the CMV promotor contained in the plasmid vector allows the viral antigen to be produced in the environment of the infected cells. Some antigen produced by the infected cells is subsequently processed and presented on the cell surface as T cell epitopes, whereas other antigen is secreted as free antigen. Immunization is effected directly by the infected cells as well as antigen secreted by the cells.

The formulation of ODV and its mode of operation is fundamentally different from that of other vaccines in that immunization is brought about by infection. Immunization achieved by this means is more akin to the way immunity is naturally acquired than with other vaccines and as a result, has a greater likelihood of mobilizing a more extensive range of components of the immune system in the process. As will be shown in subsequent examples, an outstanding feature of ODV is its capacity to evoke a more vigorous CMI response than can be achieved by conventional vaccines, and it is the only composition known to-date that can also effect long term suppression of HBV expression in transgenic mice.

### Example 2

### Antigenic Activity of ODV Expressed by Macrophage

Efficacy of the oral composition was first assessed in experiments carried out in Balb/C mice. One type of experiment was designed to assess the capacity of the oral composition to infect peritoneal macrophages cells and the extent to which the vaccine gene is expressed, processed and presented by the infected cells. In these experiments, peritoneal macrophages were harvested as adherent cells from these animals according to standard protocol. The cells were infected with the vaccine bacteria or control bacteria carrier at a ratio of 10 bacteria to 1 macrophage. The cells were washed, treated with 50 μg per ml of gentamycine to kill the residual extra cellular bacteria and then incubated for 16 hours in medium containing 10 μg per ml of tetracycline to inhibit bacterial growth. As a comparison, the cells were incubated for 2 hr with 10 μg per ml of purified HBsAg. An additional control included the P815 cell line and the same cell line transfected with the HBsAg gene (P815-S).

Table 1 shows that high levels of the viral antigen were detected in the culture supernatant and cytosol of P815-S, and the cells showed positive immunostaining for the viral antigen. On the other hand, the viral antigen was detected at low levels in the cytosol of macrophages infected with the oral composition (M-ODV), but not in the culture supernatants, and the cells showed a negative immunostaining for the viral antigen. The control cells, including macrophage infected with the bacterial carrier (M-BC) or treated with phytohemeagglutinin (M-PHA) and the parental 815 cells (P815), were similarly tested and gave a negative immunostaining for the viral antigen, and did not show detectable expression or secretion of the viral antigen.

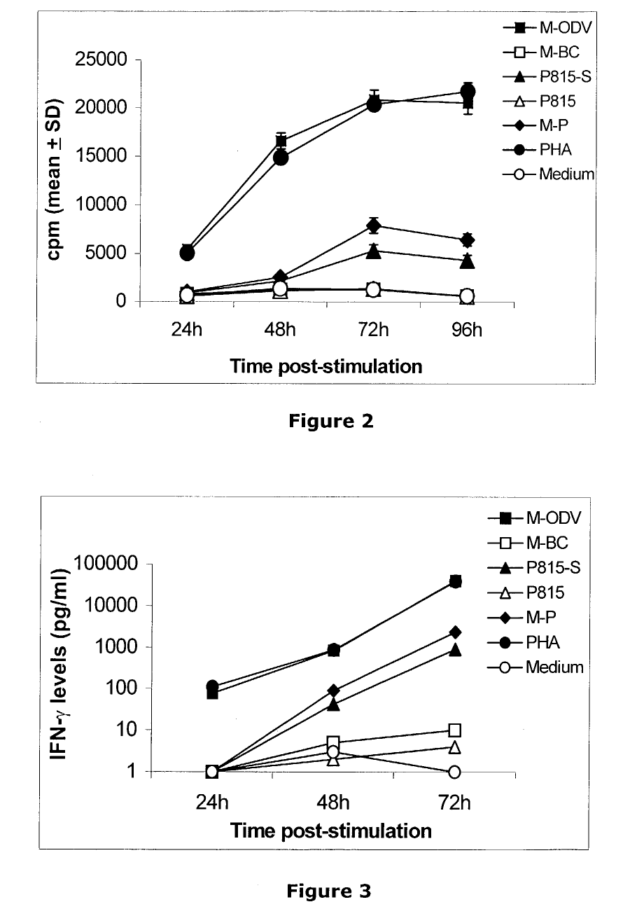

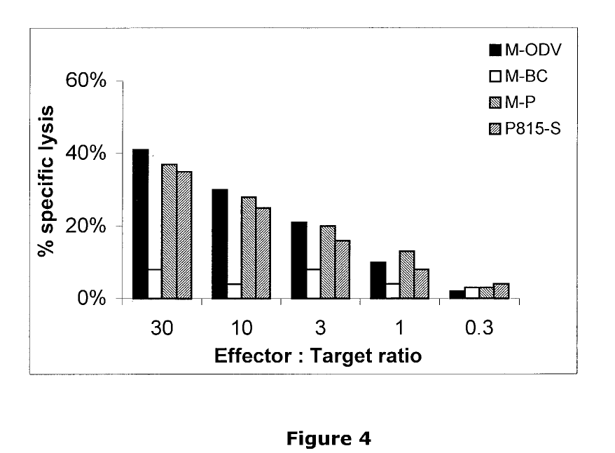

TABLE 1
Expression and presentation of oral DNA composition by
peritoneal macrophages.

| | Expression | | | Presentation of T cell epitopes^{a} | | | |
|---|---|---|---|---|---|---|---|
| | ELISA^{b} | | Immune | Prolifer- | | | |
| | ng/ml | | staining | ation | IFN-γ | IL-4 | CTL |
| | Lysate Supernatant | | | SI | pg/ml | pg/ml | m E:T |
| M-ODV | 2.8 | — | — | 16.7 | 39165 | 2 | 3 |
| M-BC | — | — | — | 1.1 | 10 | 0 | >30 |
| M-P | NA | NA | NA | 6.3 | 2318 | 2 | 3 |
| P815-S | 870 | 328 | 0 | 4.2 | 890 | 1 | 10 |
| P815 | — | — | — | 1.1 | 4 | 0 | NA |
| PHA | NA | NA | NA | 16.3 | 38235 | 1 | NA |
| Peritoneal MF were infected with a live oral DNA composition (M-ODV) or the carrier bacteria (M-BC). The contents of HBsAg in the cell lysates and culture supernatants were determined by ELISA. Immunostaining using a HBsAg specific antiserum was carrier out on cell smears to assess reactivity of these cells for the viral anitgen. Presentation of the HBsAg specific T cell epitope by these cells were assessed by the capacity of these cells to stimulate splenocytes from | | | | | | | |
| # Balb-C mice previously immunized with HBV DNA vaccine by the following assays: These are the cell proliferation assay as described in FIG. 2; the assays for HBsAg induced elaboration of gamma interferon and IL-4, respectively, as in FIG. 3; and the HBsAg specific cytotoxicity assay in FIG. 4. The results were compared with the effects of MF, which had been loaded with purified HBsAg by incubating these cells with 10 mg/ml of purified protein (M-P); P815 cell line and | | | | | | | |
| # P815 cell that harbors the same as contained (P185S) S; as well as the effects of a T cell mitogen, PHA, on the same immune splenocytes. | | | | | | | |

The M-ODV was tested in further experiments to determine if it processes and presents the viral antigen to immune T cells. The immune cells used in these experiments were derived from spleen cells of mice, which were previously immunized with 3 doses of 100 μg of the vaccine plasmid each. The DNA vaccine was given intramuscularly to the tibialis anterior muscle at 3 weekly intervals, and the animals were sacrificed 2 weeks after the final dose. FIG. 2 showed that recognition of antigen presented by M-ODV stimulated a vigorous proliferation of the immune cells to an extent that was comparable to the stimulatory effects of the T cell mitogen, phytohemeagglutinin (PHA), and surpassed the stimulatory effects of P815S and macrophages that had been loaded with purified recombinant HBsAg (M-P). While the M-BC and P815 were not stimulatory, proliferation of immune spleen cells treated with these cells was similar to the unstimulated medium control.

Recognition of the viral antigen by the Th1 subpopulation of immune T cells was determined by the gamma IFN elaboration assay as shown in FIG. 3. The results showed that M-ODV and PHA stimulated a vigorous elaboration of cytokine, while P815 and M-P showed moderate stimulatory effects. The controls were not stimulatory for the immune spleen cells.

Recognition of the viral antigen by cytotoxic T cells (CTL) was determined by the CTLp assay shown in FIG. 4. In this assay, CTL precursors contained in the immune cells were first stimulated to proliferate and differentiate into functional cytotoxic cells. Target cells previously labeled with Calcein AM were then mixed with a graded number of the stimulated immune cells (effector). The level of cytotoxicity is measured as the lowest number of the stimulated immune cells required to effect 20% specific lysis of the target cells. The results show that immune cells which had been stimulated with M-ODV, M-P or P815-S are similarly cytotoxic for the P815-S target and those stimulated with M-BC were not cytotoxic, suggesting that the stimulation was specific for HBsAg.

Table 1 summarized expression and presentation of HBsAg by mouse peritoneal macrophages infected with ODV. The results confirmed that the vaccine gene was delivered into macrophage by the carrier bacteria through a process of active infection, and that the macrophage had undergone autolysis thereby releasing the viral gene. It would appear that the viral antigen expressed was efficiently processed and presented as T cell epitopes. Consequently, the infected cells stimulated a vigorous proliferation of immune T cells to an extent that was comparable to the stimulatory effect of the common T cell mitogen, PHA. The infected cells were recognized also by HBsAg specific Th1 cells and CTLp, stimulating an active elaboration of gamma IFN by the former and the development of functional HBsAg specific CTL by the latter. On the other hand, they did not appear to be stimulatory for Th2 as to induce elaboration of IL-4. Moreover, it would seem that while most of the viral antigens expressed were processed through the Th1 pathway, free antigen was detected only at low levels in the cytosol, but there was no detectable secretion of the antigen in the culture supernatants. This was in contrast to P815-S, which actively secreted the viral antigen in the culture supernatant, whereas processing and presentation of which as T cell epitopes appear to be less efficient than M-ODV. Consequently, free antigen was detected at high levels in cytosol and culture supernatants, but these cells were less stimulatory for the immune T cells. The stimulatory effects of M-P on immune T cells and the different subpopulations were intermediate between M-ODV and P815-S.

In summary, results of the above-described experiments highlight the following features regarding the ODV: (1) the carrier bacteria provides an effective means to deliver and release the vaccine plasmid into host cells; (2) subsequent expression of the vaccine gene in the eukaryotic environment of the host cells into HBsAg is under the direction of the CMV promotor; and (3) the activity of the endogenously expressed antigen depends on the type of host cells. Thus, in professional antigen presenting cells, such as macrophage and dendritic cells, the expressed antigen is presented mainly as T cell epitopes, while in other types of cells, such as P815-S, a substantial proportion of it exists as free antigen and is secreted. Macrophage can also take up exogenous antigen and process and present it as T cell epitopes, but the level of the antigenic activity was lower than that derived from the endogenously expressed antigen.

### Example 3

### ODV Evokes Vigorous Th 1-Type Immune Response in Mice

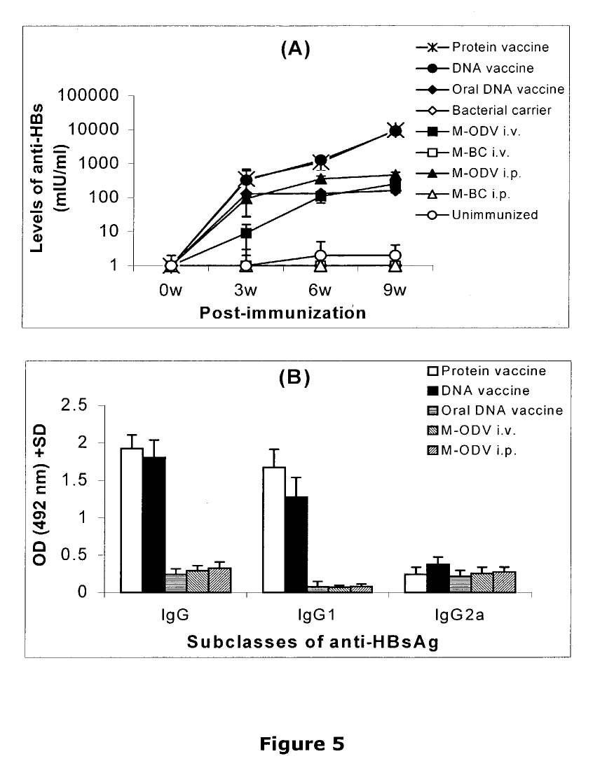

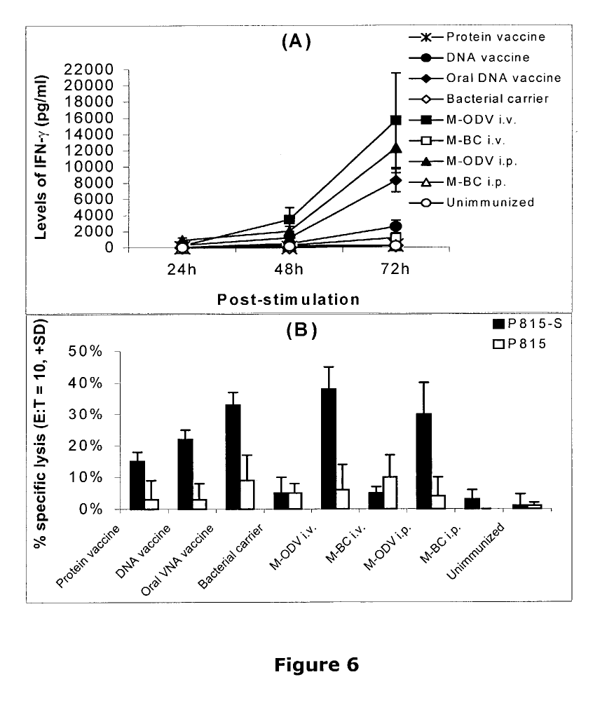

The capacity of the ODV to evoke an immune response was assessed in immune competent Balb C mice and compared with the DNA vaccine (pRc/CMV-HBs(S)) and a commercial recombinant protein vaccine (HB-VAXII, MSD, USA). The animals were studied in groups of five. The oral DNA composition was administered by feeding animals with 3 doses of 6×10⁹ of the vaccine bacteria or control bacteria at 2 days intervals, or by infusing intravenously and to the peritoneum 6×10⁷ M-ODV or M-BC as described in Example 2, three times once every 3 weeks. The other groups of animals were injected intra-muscularly to the tibialis anterior muscle with three doses of either 2 μg of the protein vaccine or 100 μg of the DNA vaccine each once every three weeks. The DNA vaccine comprised the same vaccine plasmid as contained in the ODV. Blood samples were taken every three weeks for the determination of antibody against HBsAg (HBsAb) (FIG. 5). The animals were sacrificed on week 9, three weeks after the final dose of immunization. Spleen cells taken from these animals were tested for HBsAg specific Th1 cells and CTL by the IFN-g induction assay (FIG. 6A) and cytotoxicity assay against the P815-S and P815 targets cells (FIG. 6B), respectively.

The results showed that the protein and the DNA vaccine evoked a vigorous antibody response in these animals and the level of the antibody was increased after each booster with these vaccines (FIG. 5A). The antibody produced in response to these vaccines was dominated by the IgG1 isotype (FIG. 5B). The oral DNA composition administered by feeding or via the infected macrophages, on the other hand, evoked a moderate response in these animals. The levels of the antibodies reached at 9 weeks post immunization were about 10 times lower than that, which were produced in response to the protein and DNA vaccine, and the antibody produced were predominantly IgG2 isotype. On the other hand, the oral DNA composition, either administered by feeding or through macrophage, evoked a vigorous Th1 as well as CTL response in these animals. The level of Th 1 response reflected by the amount of IFN-γ elaborated (FIG. 6A) and that of CTL response shown as percent of specific lysis of the P815-S target cells (FIG. 6B) were markedly higher than that achieved by the DNA vaccine, whereas the protein vaccine evoked no detectable Th1 response nor, a significant CTL response.

The response evoked by the ODV is consistent with the view that immunization was achieved through a process of active infection caused by the carrier bacteria. It appears that, as in natural infection, the carrier bacteria preferentially infected phagocytic cells residing in the intestinal mucosa and those which had been recruited to the site in response to inflammation associated with the infection. Indeed, being an established intracellular parasite, the carrier bacteria are adept to infect such inflammatory cells. Consequently, the composition evoked a vigorous Th1 and CTL response and production of moderate level of IgG2 antibody similarly as the response brought about by infusion of macrophages infected ex vivo with ODV (Table 2).

TABLE 2
Adoptive transfer of macrophages infected with oral DNA composition
reproduced the same immune profile as that induced
by oral DNA vaccination.

| | | Anti-HBsAg | | | |
|---|---|---|---|---|---|
| | | mlU/ml | IFN-γ | IL-4 | CTL |
| | | Mean ± SD | pg/ml | pg/ml | m E:T |
| Profiles | Immunogens | (predominant subtype) | Mean ± SD | Mean ± SD | Median |
| 1 | Protein Vaccine | 9632 ± 413 (IgG1) | 256 ± 167 | 13 ± 3 | 30 |
| 2 | DNA Vaccine | 9250 ± 948 (IgG1) | 2587 ± 771 | 13 ± 5 | 10 |
| 3 | Oral DNA | 160 ± 24 (IgG2a) | 8272 ± 1423 | 11 ± 3 | 3 |
| | Composition | | | | |
| 3 | M-ODV i.v. | 253 ± 7 (IgG2a) | 15690 ± 5827 | 58 ± 29 | 3 |
| 3 | M-ODV i.p. | 466 ± 94 (IgG2a) | 12302 ± 3062 | 48 ± 13 | 10 |
| 0 | Bacterial Carrier o.r. | <4 | 197 ± 140 | <1 | >30 |
| 0 | M-BC i.v. | <4 | 1206 ± 140 | 14 ± 7 | >30 |
| 0 | M-BC i.p. | <4 | 251 ± 65a | 10 ± 8 | >30 |
| 0 | Unimmunized | <4 | 199 ± 41 | 10 ± 2 | >30 |
| Nine groups of five mice each were immunized as follows: Two groups were injected i.m. with the protein or DNA vaccines. Two groups were orally given the oral DNA composition or bacterial carrier. Four groups were respectively infused i.v or i.p with MF infected with the oral DNA composition (M-ODV i.v. or i.p.) or with the bacterial carrier (M-BC i.v. or i.p.). The last group served was not immunized. Antibody response was determined as described in FIG. 5 and | | | | | |
| # T cell response, as in FIG. 6. | | | | | |

Comparison with the responses to the other vaccines as in Table 2 suggested that efficacy of ODV was attributed largely to a combination of two features pertaining to its formulation. The first is that the carrier bacteria which preferentially targets the vaccine gene to the APC, and the second is the CMV promoter contained in the plasmid vector which enables the viral antigen to be produced endogenously by these cells. The importance of targeting the APC was evidenced by comparison with the DNA vaccine administered intra-muscularly. In the latter instance, the vaccine gene was likely taken up largely by muscle cells, which being non-professional antigen presenting cells, expressed and actively secreted the viral antigen, thereby evoking production of a high level of IgG1 antibody as did the protein vaccine. The DNA vaccine, however, was less efficacious in evoking a Th1 type immune response than the ODV, where the endogenously expressed antigen was processed and presented by the APC.

The following provides a more detailed discussion of the Examples 2 and 3. In a previous study, we had shown that live oral vaccination with Salmonella typhimurium delivering plasmid DNA-HBsAg (oral DNA vaccine) evoked a vigorous T cell response and a weak antibody response with predominant subclass IgG2a in mice, suggesting a significant involvement by professional antigen presenting cells (APC). In the present study, this possibility was further studied by infecting peritoneal macrophages (Mφ) with the oral DNA vaccine. Although, the infected cells could only express low level of the viral antigen, they nevertheless stimulated a vigorous lymphocyte proliferation of splenocytes from immune mice, induced these cells to elaborate interferon-γ and stimulated development of HBV-specific cytotoxicity against target cells expressing the viral antigen. Infusion of the infected Mφ evoked a vigorous Th 1 and cytotoxic T lymphocyte (CTL) response and a weak IgG2a antibody response in mice, which was essentially the same as response to the oral DNA vaccine. In contrast, recombinant protein vaccine evoked a vigorous IgG1 antibody response and a weak T cell response. While, given intramuscularly, the same plasmid DNA vaccine as that contained in the oral DNA vaccine evoked a vigorous IgG1 antibody response and a moderate T cell response in these animals. It was concluded that professional APC may orchestrate the immune response to live oral DNA vaccine and it was of interest to note that different vaccine formulation and routes of administration evoke distinct immune response to HBV.

Materials and Methods

Mice

BALB/c mice (H-2^{d}) were bred under standard pathogen-free conditions in the laboratory of animal unit of the University of Hong Kong. Female mice of 4-6 weeks of age, weight 14-16 g were used in this study. The criteria outlined in the “Guide for the Care and Use of Laboratory Animals” (NIH publication 86-23, 1985) were followed.

Cell Lines

The P815 cell line (TIB-64) was obtained from the American Type Culture Collection (USA). The P815 cell line with stable HBsAg expression (P815-S) was kindly provided by Reimann and coworkers. Both cell lines were cultured in MEM (Gibco-BRL, USA), supplemented with 10% FCS and antibiotics, but for the latter, the medium also containing 1 mg/ml of G418 (Sigma, USA).

Bacterial Strain and Plasmid

S. typhimurium aroA strain SL 7207 (kindly provided by Stoker) was used as carrier for the in vitro and in vivo studies. Plasmid pRc/CMV-HBs was a generous gift from Whalen and coworkers 15 and was used for the transformation of S. typhimurium (oral DNA vaccine) and for intramuscular immunization (DNA vaccine).

Mφ ex vivo Infection and Antigen Loading

Each BALB/c mice was peritoneally injected with 100 μg of Conconavlin A (Sigma, USA) in 1 ml serum-free RPMI (0% RPMI, Gibco-BRL, USA). Primary peritoneal Mφcells were harvested by washing the mouse peritoneum using 30 ml syringe/20 G needle with 10 ml of antibiotic-free 0% RPMI 3 days thereafter. The Mφ were incubated in 6-well plates containing 2×1 cells per well at 37° C. for 2 h. After removing the non-adherent cells, the Mφwere infected with oral DNA vaccine or its bacterial carrier S. typhimurium at MOI 10 to yield M-ODV and M-BC, respectively. The remaining extracellular bacteria were killed by adding 50 μg/ml of gentamicin in RPMI supplemented with 10% FCS (10% RPMI) 30 min thereafter and incubated for 4 h. The intracellular bacteria multiplication was inhibited by an additional over night incubation in the presence of 10 μg/ml of tetracycline. The Mφ were also loaded with 10 μg/ml of protein HBsAg and incubated for 2 h to generate M-P. These Mφ cells were treated with 10 mM of EDTA for 5-10 min at 4° C. and the detached cells were harvested.

Schedule of Immunization and Adoptive Transfer

Around 45 mice were divided into nine groups (5 mice per group). Two groups were injected intramuscularly (i.m., tibialis anterior muscle) with either 2 μg per dose of protein HBsAg vaccine (HB-VAX II, MSD, USA) (protein vaccine) or 100 μg per dose of DNA vaccine for three times at 3-week intervals. Two other groups were given three doses of 6×10⁹ per mouse of oral DNA vaccine or bacterial carrier by oral route at 2-day intervals. Adoptive transfer of 5×10⁷ per dose of M-ODV and M-BC was administered either intravenously (i.v.) or intraperitoneally (i.p.) for three times at 3-week intervals. The unimmunized mice served as negative controls.

Detection of HBsAg Expression in Infected Mφ

Culture supernatant and cell lysates at 5×10⁷ cells/ml of M-ODV or M-BC cells were subjected to the HBsAg detection by ELISA (BIOKIT, SA, Spain) according to the manufacturer's instruction. Culture supernatants and cell lysates of P815-S and P815 cells were applied in the experiments as controls. HBsAg levels were quantified using a panel of HBsAg calibrators (3.769-0.248 ng/ml) provided by Abbott Diagnostics (USA). The expression of the antigens in individual cells were also tested by immune staining using anti-HBsAg immune staining kit (DAKO, USA) according to standard procedure.

Detection of Serum Anti-HBs

Sera were obtained from the mice before and after immunization at 3-week intervals. Anti-HBs was determined by ELISA (BIOKIT, Spain) according to the manufacturer's instructions. Antibody levels were quantified using standard positive controls (10-100 mIU/ml) provided by the kits. The subclasses of these antibodies were identified by ELISA using the same kit, but HRP-conjugated sheep anti-mouse IgG, IgG1 and IgG2a (SeroTec, UK) were used instead.

Proliferation Assays

The spleen cells were obtained from three mice immunized with three doses of DNA-HBsAg vaccine at 9-week post-immunization and suspended in 10% RPMI. The pooled splenocytes (5×10⁵ per well) were, respectively, mixed with M-ODV, M-BC, M-P, irradiated (20,000 rad) P815-S and P815 at an effector:stimulator ratio of 20, 25 μg/ml of PHA or culture medium alone. The mixtures were cultured in triplicate wells at 37° C. in 96-well microplates. After 24, 48, 72 and 96 h of incubation, the cells were labeled for 16 h with 1 μCi of [³H] thymidine per well. The DNA incorporating radioactivity was measured thereafter by a scintillation counter. Results were expressed either as mean counts per minute (cpm) of triplicate cultures or as the stimulation index (SI), which was calculated as the ratio between mean cpm obtained in the presence and absence of stimulator.

Antigen-induced Cytokine Secretion Assays

Production of HBsAg-induced IFN-_{.}γ and IL-4 in the same cultures as described above were detected by ELISA using Opt EIA kits (PharMingen, USA) according to the manufacturer's instruction. Levels of IFN-γ_{.} and IL-4 in supernatant obtained at 24, 48 and 72 h post-stimulation were quantified using at least six concentrations of standard IFN-γ_{.} and IL-4 provided by the kits.

CTL Assays

Spleen cells obtained from individual mice were, respectively, stimulated with M-ODV, M-BC, M-P and irradiated P815-S at effector:stimulator ratio of 20, or 10 μg/ml of purified protein HBsAg (Research Diagnostics Inc., USA) for 3 days. The specific CTLs were expanded thereafter by adding 25 IU/ml of murine rIL-2 (R&D systems, USA) for an additional 4-5 days. The CTL activity in the cultures was measured in triplicate in a standard 4 h calcein release assay in U-bottom 96-well microplates. The cytolysis of the targets was determined by measuring Calcein AM (Molecular Probes Inc., USA) fluorescence intensity (FI) using a fluorometer. The percentage-specific cytolysis (5) was calculated as follows:

$$( 1 - \frac{experimental FI - total lysis FI}{target control FI - total lysis FI} ) \times 100 \%$$

Results

HBsAg Expression and Presentation by Mφ Infected with Oral DNA Vaccine.

Peritoneal Mφ were infected with the oral DNA vaccine (M-ODV) and its bacterial carrier control (M-BC), or loaded with 10 μg/ml of purified protein HBsAg (M-P). The P815-S and P815 cells were also included as additional controls. Expression of viral antigen by the infected Mφand P815-S was determined by ELISA and immunocytology. Processing and presentation of the expressed antigens by these cells were accessed by their stimulatory effect to splenocytes from syngenic BALB/c mice previously immunized i.m. with three doses of DNA vaccine.

In agreement with earlier finding, the P815-S cells could efficiently express HBsAg, which was detected in culture supernatant and cell lysate. The in vitro infection of Mφ by oral DNA vaccine resulted in detectable HBsAg reaching 2.8 ng per 5×10⁷ cell lysate on day 3 post-infection, by the level of HBsAg expressed in these cells was at least 300-fold lower than those in P815-S. Furthermore, these cells did not elaborate detectable amount of the viral antigen in the culture supernatant and the antigen expression was too low to be visualized by immune staining (results not shown).

The presentation of viral antigen in M-ODV cells was studied by their stimulatory effect to immune T cells, which was determined by the lymphocyte proliferation, cytokine induction and CTL assays. The results were compared with the stimulatory effects of M-P, P815-S, and the non-specific stimulant PHA.

The lymphocyte proliferation assay showed that the M-ODV cells stimulated a vigorous proliferation of splenocytes from the immunized mice. The stimulatory effect of these infected Mφ was similar to that of the T cell mitogen, PHA, and surpassed that of M-P or P815-S, respectively, while the control M-BC and P815 cells did not show a stimulatory effect when compared with the medium control (FIG. 2).

The stimulatory effects of M-ODV cells to Th 1 and Th 2 cells were further assessed by the elaboration of IFN-γ_{.} and IL-4. Elaboration of IFN-γ_{.} was detected in the splenocyte culture after stimulation with M-ODV for 24 h (FIG. 3). The production of IFN-γ_{.} thereafter reached a highest level similar to that induced by PHA, which was about 17- and 40-fold higher than those in the cultures stimulated by M-P and P815-S, respectively. The responses were HBsAg-induced as all the control cultures stimulated by M-BC and P815 did not induce significant production of IFN-γ_{.}. The same culture supernatants were also assayed for IL-4. However, presumably the binding of the cytokine to its receptors on the splenocytes (Doherty, personal communication), the level of IL-4 presented in the culture supernatants was too low to reflect the stimulatory effect (data not shown).

The CTL assay was performed by co-culturing the splenocytes with M-ODV, M-BC, M-P and irradiated P815-S cells for 7 days to stimulate CTL precursors to proliferate and differentiate into functional CTLs. Graded number of Calcein AM-labeled P815-S or its control P815 targets were then added to the cultures at the effector to target (E:T) ratios of between 0.3 and 30. The CTL activity in the cultures was determined by a 4 h CTL assay (FIG. 4). The results showed that the splenocytes stimulated by M-ODV exhibited a high level of cytotoxicity against P815-S. The observed cytotoxicity was directed specifically against the viral antigen presented by the target cells, and the stimulated splenocytes were not also cytotoxic to the control P815 target, which do not express the viral antigen. The level of specific cytotoxicity was lower for splenocytes stimulated with irradiated P815-S and the effect was similar to that due to M-P. Splenocytes stimulated with M-BC did not develop detectable HBsAg-specific cytotoxicity.

Taken together, the results suggest that, in Mφ infected with oral DNA vaccine, the insufficiently expressed viral antigen is efficiently processed and presented by these cells in association with MHC I and MHC II Molecules. Consequently, these cells, but not also the control Mφ infected with the same strain of carrier bacteria, were strongly stimulatory for the subsets of Th 1 and CTL cells (Table 1).

Immune Responses to Adoptive Transfer of MφInfected with Oral DNA Vaccine and HBV Vaccination

To assess the immunogenicity, M-ODV cells were infused i.v. or i.p. to BALB/c mice. The ensuing B and T cell responses were compared with those observed in three groups of immunized animals and four groups of controls. These included those, which were orally given the oral DNA vaccine and i.m. administered DNA or protein vaccines. Additional four groups of animals served as controls, which were infused i.v. or i.p. with M-BC, orally given the bacterial carrier and unimmunized.

The DNA and protein vaccines elicited a vigorous antibody response (FIG. 5A) with predominantly IgG1 subclass (FIG. 3B). Antibody level produced in the animals, 9 weeks after immunization were over 20 times higher than those in the other group. The antibody response to the oral DNA vaccination was similar to that seen after i.v. or i.p. infusion of the M-ODV (FIG. 5A) and the antibody produced by these animals was mainly of the Ig2a subclass (FIG. 5B). All four groups of control animals did not show detectable-specific antibody response.

To study T cell responses, splenocytes obtained from the animals, 9 weeks after immunization were cultured at concentration of 5×10⁶ cells/ml in the presence of 10 μg/ml of purified HBsAg for 3 days. Aliquots of the culture supernatants were taken at 24, 48 and 72 h post-culture and analyzed for antigen-induced cytokines. The cultures were further incubated for 4-5 days in the presence of additional 25 IU/ml of murine rIL-2 and tested for cytotoxicity against Calcein AM-labeled P815-S and its control P815 targets. The results showed that i.v. or i.p. infusion of M-ODV, as did the oral DNA vaccination, elicited a vigorous Th 1 response, which was evidenced by the vigorous IFN-γ_{.} production (FIG. 6A), and CTL response (FIG. 6B). The responses to the DNA i.m. vaccination were weaker. The protein vaccine evoked an equivocal CTL response, but undetectable Th 1 response. Unlike the secretion of IFN-y_{.}, there was neither significant difference between groups nor obvious increase of IL-4 production following specific stimulation (data not shown).

The results described above showed that immune response of the animals is subject to influence by vaccine formulation and routes of immunization (Table 2). The response to conventional protein vaccine is characterized by a vigorous IgG1 antibody (Th 2) response, accompanied by weak Th 1 and CTL responses (profile 1). The DNA vaccine given i.m. evoked a similarly vigorous IgG1 antibody production as the protein vaccine and a moderate T cell response (profile 2). In contrast, the same DNA vaccine, when delivered orally via the carrier bacteria, evoked vigorous Th 1 and CTL responses, but a weak antibody production dominated by the IgG2a subclass (profile 3). Importantly, the response to the live oral DNA vaccine is essentially the same as that observed following infusion of Mφ infected with the oral DNA vaccine. This suggests that the response to the oral DNA vaccine may be orchestrated by professional APC, such as Mφ and DC.

Discussion

We have determined the antigenicity and immunogenicity of peritoneal Mφ, which had been infected with a live oral DNA vaccine, to assess the role of APC in immune response to the vaccine. A crucial role of Mφplaying in this strategy of immunization was not only evidenced by that Mφ can express, process and present exogenous viral antigen, but also further confirmed by that both i.v. and i.p. infusions of Mφ ex vivo infected with oral DNA vaccine may essentially reproduce the same immune profile as that seen by the oral DNA vaccination.

The oral DNA vaccine comprises S. typhimurium aroA strain SL 7207 harboring the plasmid DNA-HBsAg. The carrier bacterium has a mutation which causes it to undergo autolysis, after it has been ingested by the cells [18]. Expression of the released DNA vaccine was evidenced by detection of a small amount of intracellular viral antigen in lysates of the infected cells. However, the infected cells did not secrete detectable amount of the free antigen in the culture supernatants and the amount of the antigen expressed was too low for it be directly visualized in the infected cells by immunocytology. This was attributed, in parts at least, to efficient processing and presentation of the expressed antigen by these professional APC. Although, not reactive with the HBsAg antibody, the processed peptides were nevertheless presented by the infected Mφand recognized in association with MHC molecules by immune T cells. Consequently, the infected cells stimulated a vigorous lymphocyte proliferation, when they were co-cultured with splenocytes from syngenic mice previously immunized i.m. with the DNA vaccine. The infected Mφalso induced elaboration of IFN-γ_{.} by Th 1 cells and stimulated HBsAg-specific CTLs. These findings further suggest that the processed antigen was recognized by immune Th cells in association with MHC class II molecules and by CTLs in association with MHC class I molecules. Compared with the infected Mφ, P815-S cells and Mφloaded with protein HBsAg were less efficient stimulant of HBsAg-specific lymphocyte proliferation and Th 1 response (Table 1). A characteristic feature of S. typhimurium is that it remains confined to a membrane-bound compartment and is thus insulated from the cytosolic environment after invading the host cells. The poor expression of exogenous HBsAg followed by excellent processing and presenting in the infected Mφ suggested that the remains of lysed Salmonella might be a strong adjuvant for presenting antigen in association with MHC molecules to stimulate the Th and CTL responses.

Infusion of the infected Mφwas found ot evoke a vigorous Th 1-type immune response in the animals. The response was evidenced by a weak induction of antibody dominated by the Th 1-dependent IgG2a subclass, vigorous antigen-induced IFN-_{γ}. production and development of antigen-specific cytotoxicity. This was essentially the same response as that observed after oral DNA vaccination. It thus highlights an important role of the APC in orchestrating the immune response. Previous study suggests that the response is brought about by a process of infection. The accompanying inflammation would seem to have ensured an efficient uptake of the ingested bacteria by professional APC via the phagocytosis and transcytosis. The involved APC may include those Mφ and DC which were initially presented in the intestinal mucosa and those, which had been subsequently recruited to the site of infection. The immune response was probably initiated upon drainage of the infected APC into the regional lymph node in the Peyer's patch, and was dominated by a vigorous Th 1 cell and CTL response similar as that effected by infusion of peritoneal Mφinfected ex vivo with the oral DNA vaccine.

It was of special interest to note that the same plasmid DNA vaccine administered i.m. had evoked a distinct immune profile, which is intermediate between the Th 1-type response that was orchestrated by oral DNA vaccination and the Th 2-type response evoked by the protein vaccine. It may be probably ascribed to the extent of APC involvement and released free antigen in this type of immune response. The DNA vaccine i.m. immunization neither induce an overt inflammation nor results in enrichment of phagocytic APC. Unlike a transient presence of large amount of antigen resulting from protein vaccination, the antigen expressed by DNA in the muscle cells may be gradually released into the circulation and probably sustained for a protracted period. The released antigen may be taken up by APC to prime Th 1-type response, or amplify B cells to produce antibody via Th 2 pathway. In contrast, the predominant Th 1-type responses to the same DNA vaccine delivered by Salmonella seems to be preferably triggered by the infected APC but not the free antigen. The phagocytic APC can be quickly recruited to the infection site, express and present the foreign gene carried by the bacteria to orchestrate this type of immune response. It would seem on the other hand that, apart from the professional APC, the other cell types infected by the oral vaccine did not secrete adequate amount of the viral antigen to stimulate production of IgG1 antibody. Consequently, the oral vaccine, as did the infected macrophages, evoked only a weak antibody response, dominated by the IgG2a subclass, in these animals. Thus, different formulation of the same plasmid DNA vaccine may induce distinct immune response.

Chronic HBV infection, which is a major health issue in many parts of the world, is attributed to T cell immune tolerance to the virus. The tolerance could be broken, leading to permanent clearance of HBV from patients with chronic HBV infection, by adoptive transfer of immunity from donors who had acquired the immunity from natural HBV infection. Previous studies had shown that vaccination with the HBsAg-anti-HBs immune complex (IC) could effect HBsAg seroconversion and clearance of serum HBV DNA, in the patients. In a HBsAg transgenic mouse model, we had also shown that the immune tolerance to the transgene could be also broken efficiently by immunization with IC+DNA-HBsAg, which was similar to those observed by other investigators in HBV transgenic mice after receiving adoptive cytokine-activated DC [23]. Presumably, the therapeutic effect of IC+DNA was achieved by enhancing uptake of the vaccine by APC via their Fc receptors and through efficient engulfment of the particulate antigen. The present study has exploited a crucial role of phagocytic APC in oral DNA vaccination, which can efficiently elicit strong HBsAg-specific Th 1 and CTL responses. It is thus reasonable to assume that this strategy of immunization may contribute towards breaking immune tolerance in chronic HBV infection. Further study is underway to determine whether this type of oral DNA vaccination can indeed break the immune tolerance to trigger an antigen-specific, T cell-mediated immune response in the HBV transgenic mouse model.

### Example 4

### A Single Dose of ODV Induced Protracted Suppression of Transgene Expression.

In the immune competent mice, it was shown that ODV evokes a vigorous Th 1-type response characterized by high level of HBV specific CTL and Th 1 cell responses and moderate level of IgG2a antibody. On the other hand, the protein vaccine evoked production of high level of IgG1 antibody, but not also a significant cell mediated immune response. While the response to the DNA vaccine was intermediate between the two, featuring production of high level of antibody that was predominantly IgG1 and a moderate CTL and Th1 response. In this section, we shall further disclose that only the oral DNA composition and the vigorous Th1 type immune response it evokes can suppress transgene expression in the HBsAg transgenic mice.

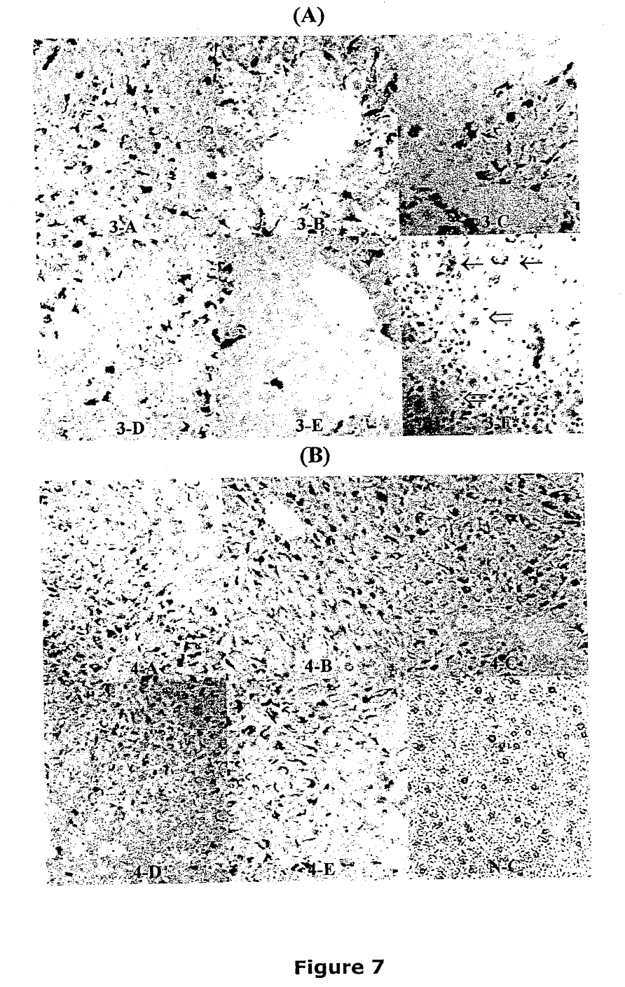

The strain of transgenic mice used was C57BL/6J-TgN (A1B1HBV) 44Bri mice (H-2^{b}). The animals were obtained from Jackson Laboratory (USA) at 8-12 weeks of age weighing 16-18 g. These animals harbor and express the HBsAg gene in the liver and kidney [21]. Expression of the transgene during embryonic stage had apparently induced a state of immune tolerance in these animals such that they do not produce detectable HBV specific antibody or immune T cells. A feature, such as that exemplified by the control animals shown in FIG. 7 and which appears to be a special to this particular strain of transgenic animal, was that the serum level of the viral antigen increased with age. The accumulation of the viral antigen in blood presumably was because the rate by which the antigen was produced and released into the blood exceeded the rate of its clearance therefrom.

Two sets of experiments were carried out. One using 5 groups of 5 to 6 animals each was conducted over 12 weeks duration to compare the effects of different vaccines. Two groups of the animals were fed orally with a single dose of the ODV or BC. Another two groups were injected intra-muscularly with 4 doses of the DNA vaccine or the protein vaccine at 3 week intervals and the fifth was the untreated control group. Another set of experiments designed to elucidate the short-term effects of the oral DNA vaccine over 4 weeks duration employed 2 groups of 12 animals each; one was given the ODV and the other BC.

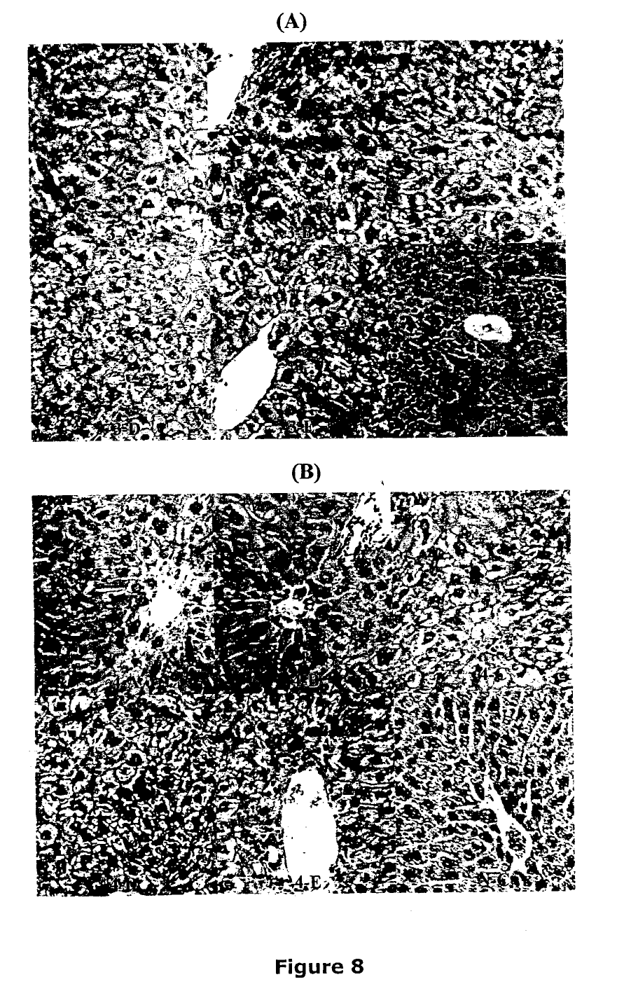

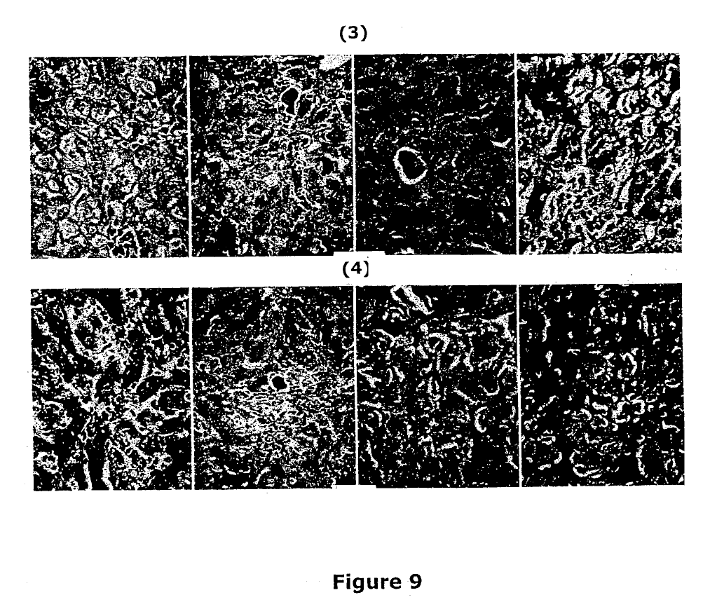

Results from both sets of experiments agreed showing that a single dose of the ODV was sufficient to suppress transgene in these animals. One animal in the first set of experiments died of fulminent hepatitis 13 days after receipt of the ODV. Mean viral RNA contents of liver tissues taken 12 weeks from the other 5 surviving animals remaining from this group was reduced by more than 4 fold compared with corresponding value determined for the control unimmunized animals (Table 3). While mean transgene contents were essentially the same for both groups of animals. Immunostaining further revealed that whereas the large majority of hepatocytes in liver sections from control unimmunized animals were immuno-reactive for the viral antigen, there was substantial but variable proportion of hepatocytes from the immunized animals that was stained negative for the viral antigen (FIG. 8). H & E staining of liver sections from both groups of the animals was normal, however, except the autopsy material taken from the animal which died of fulminent hepatitis after receipt of ODV (FIG. 9). Liver section from the latter animal showed intense lymphocytic infiltration with eosinophilic liver cell degeneration. The other vaccines did not affect transgene expression in these animals. Mean viral RNA contents of liver tissues after repeated immunization with the DNA or the protein vaccine were essentially the same as that for the control unimmunized animals or animals which were given BC (Table 3). The large majority of liver cells from these two groups of immunized animals were also reactive for the viral antigen as did liver cells of the control animals (not shown).

TABLE 3
Detection of HBsAg DNA and mRNA in liver tissues.

| | | DNA | mRNA | | | |
|---|---|---|---|---|---|---|
| | | (±SD) | (±SD) | | | |
| | | pg/mg | fg/mg | Groups | p | |
| Groups | Immunogens | liver | liver | Compared | DNA | mRNA |
| 1 | Protein Vaccine | 26 ± 15 | 91 ± 56 | 37315 | 0.495 | 0.013 |
| 2 | DNA Vaccine | 26 ± 7 | 99 ± 14 | 37316 | 0.466 | <0.001 |
| 3 | Oral DNA | 26 ± 7 | 22 ± 11 | 37318 | 0.452 | 0.004 |
| | Composition | | | | | |
| 4 | Bacterial Carrier | 26 ± 8 | 111 ± 60 | 37319 | 0.484 | <0.001 |
| 5 | Unimmunized | 25 ± 8 | 95 ± 20 | 1245 | >0.430 | >0.280 |

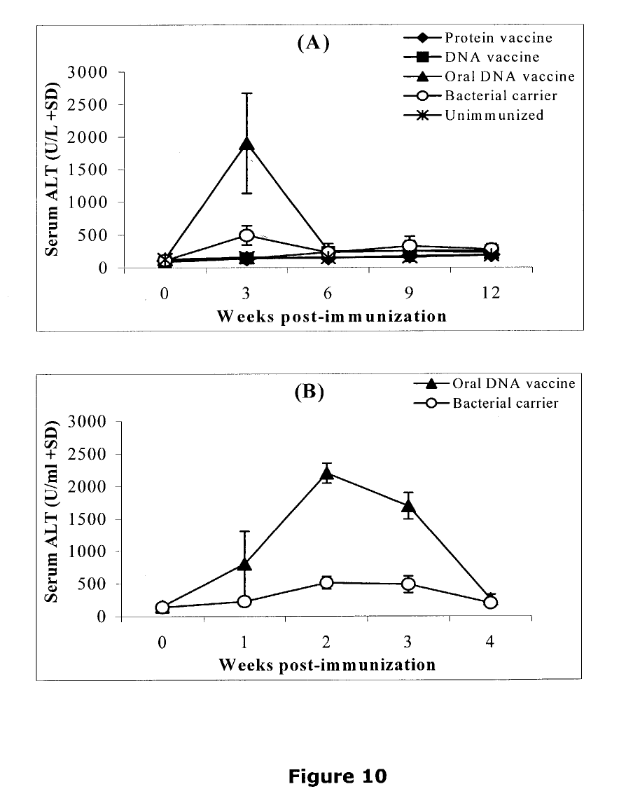

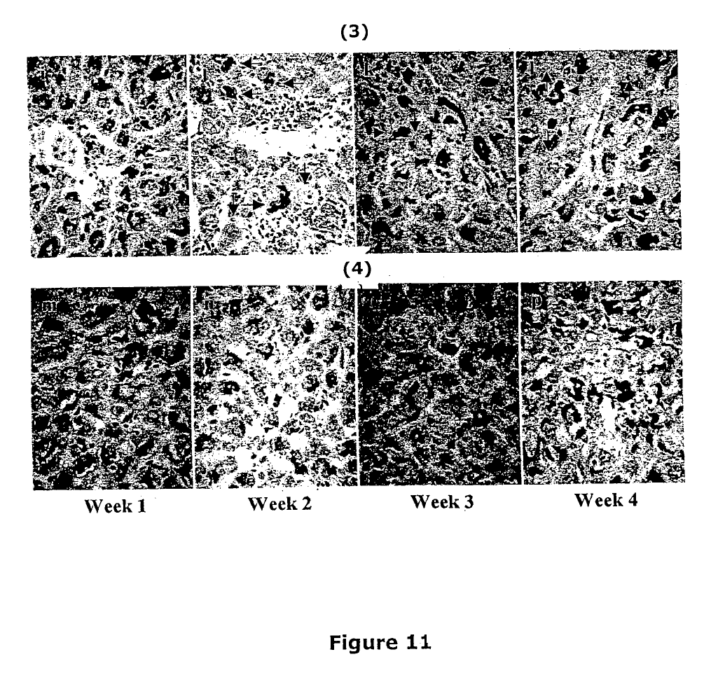

Suppression of transgene expression by the ODV was further studied in a second series of experiments. Immunostaining of liver sections taken from animals sacrificed weekly between week one and four after receipt of ODV showed that suppression of transgene expression, evidenced by decreased HBsAg reactivity of liver tissue, occurred as early as 2 to 3 weeks following immunization. In contrast to the suppressive effects observed 12 weeks after receiving the composition, early suppression was concomitant with significant liver pathology featuring focal inflammation and liver cell degeneration (FIG. 10). Serum ALT levels were also raised in samples taken on weeks 2 and 3 and returned to normal level on week 4 (FIG. 11). Liver pathology subsided on week 4 and serum ALT level also had returned to normal, but transgene expression remained suppressed in the absence of liver injury at this time as it did 12 weeks after immunization.

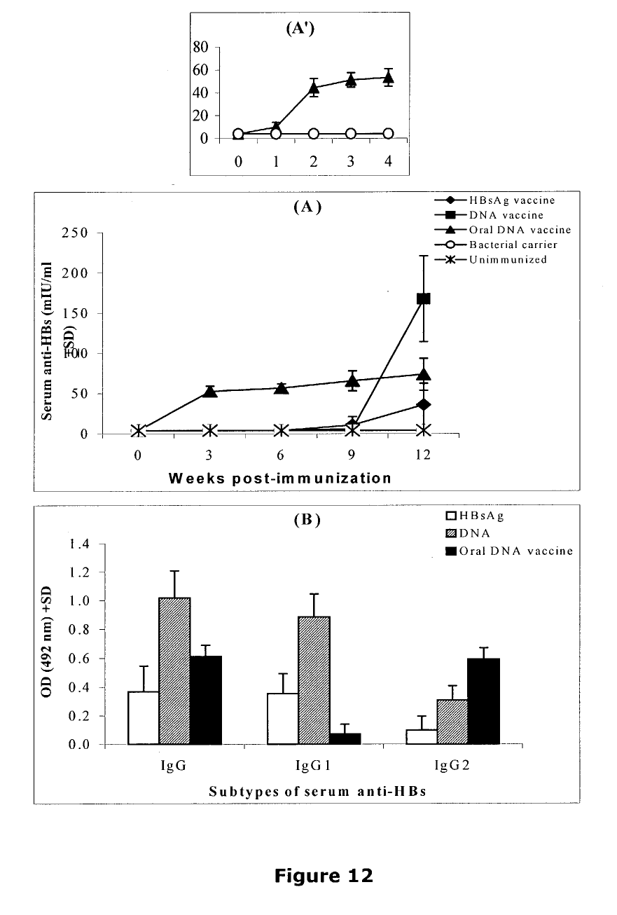

Thus, results from both series of experiments suggested that suppression of transgene was effected initially by predominantly cytopathic mechanisms accompanied by hepatic flare. Later, suppression was sustained for protracted period of time by predominantly non-cytopathic mechanisms associated with minimum pathology. The switch from the former to the latter mechanism appeared to occur about 4 weeks after receiving the ODV. The composition evoked a vigorous Th1 type response in these animals as it did in the immune competent mice described in the earlier example (Table 4). Antibody production reached peak levels after two weeks (FIG. 12) and HBV specific Th1 and CTL activity was increased markedly two weeks following receipt of the ODV when transgene suppression was first observed (Table 4). The temporal relationship suggests that suppression of transgene is likely to be primarily due to immune, rather than innate, mechanisms, although the latter are likely contributing factors. The contention is supported by results obtained with the bacteria carrier, which activates innate mechanism, but it did not also evoke specific immune response, nor suppress transgene expression.

TABLE 4
The early Th1 and CTL responses induced by
oral DNA composition in HBs-Tg mice.

| | | HBsAg- | |
|---|---|---|---|
| Weeks | | induced | CTL Activity |
| Post- | | IFN-γ | HBsAg Target Cytolysis |
| Immuni- | | Mean | (%) |
| zation | Immunogens | pg/ml ± SD | E:T = 30 ± SD |
| 1 | Oral DNA | 372 ± 54 | 24 ± 2 |
| | Composition | | |
| | Bacterial Carrier | 192 ± 49 | 17 ± 1 |
| 2 | Oral DNA | 1986 ± 164 | 45 ± 2 |
| | Composition | | |
| | Bacterial Carrier | 420 ± 64 | 21 ± 2 |
| 3 | Oral DNA | 2636 ± 335 | 43 ± 3 |
| | Composition | | |
| | Bacterial Carrier | 268 ± 65 | 23 ± 3 |
| 4 | Oral DNA | 2786 ± 513 | 38 ± 2 |
| | Composition | | |
| | Bacterial Carrier | 267 ± 27 | 16 ± 2 |

The immune status induced by the DNA vaccine, for example, which is also capable of breaking immune tolerance (albeit less effectively than the ODV), failed to suppress transgene expression when compared with the ODV, the difference being largely quantitative. This result highlights the importance of having the two features combined in the vaccine composition which makes it proficient at suppressing transgene expression. Since the transgenic mice exhibit immune tolerance prevailing in chronic infection with HBV, the finding makes the ODV an ideal vaccine candidate for the treatment of chronic infection with hepatitis B virus. More generally, it is envisaged the same combination of the two features would be important in the formulation of vaccines for treatment of other chronic viral infections, including infection with HIV.

The following provides a more detailed discussion of the Example 4. The therapeutic efficacy of oral immunization with Salmonella typhimurium aroA delivering the plasmid pRc/CMV-HBsAg (oral DNA vaccine) was compared with intramuscular immunization with the same plasmid DNA and recombinant protein HBsAg in a HBsAg transgenic mouse model. A single dose of oral DNA vaccine could break the immune tolerance versus the trangene-encoded HBsAg, resulting in vigorous Th 1-type lymphocyte responses and production of IgG2 subtype of anti-HBs. Though repeated doses of intramuscular protein or DNA vaccinations could reverse immune anergy respectively at different quantitative levels, only oral DNA vaccine down-regulated the transcription and expression of the viral transgene in hepatocytes. The level of viral mRNA in liver tissues decreased by more than 4-fold and viral antigen expression was curtailed markedly, being confined to small and scattered foci of liver sections. Moreover, the reversal of immune tolerance by oral DNA vaccine was also evidenced by an early and transient inflammatory response in liver tissue with elevated ALT in the first 3 weeks, which returned to normal level thereafter. The down-regulation at early stage appeared to be attributed to both non-cytopathic and cytopathic pathways, but it was switched thereafter to the non-cytopathic pathway. The mechanism underlying this immune strategy may involve an interplay between active bacterial infection, innate immune response including rapid recruitment of APCs and NK cells, inflammatory cytokine activation, and bacterial adjuvant effects. All these effects may have enhanced the endogenous viral antigen presentation by activated APCs and elicited potent Th 1-type of host immune response.

Materials and Methods

Mice

C57BL/6J-TgN (Alb1HBV) 44Bri mice (H-2^{b}) were provided by the Jackson Laboratory (USA). The mice were confirmed of being serum HBsAg positive. A total of fifty-two HBs-tg mice (27 males and 25 females), 8-12 weeks of age and weighing 16-18 g were used in this study. The non-transgenic C57BL/6J (H-2^{b}) and Balb/c (H-2^{d}) mice were bred under standard pathogen-free conditions in the Laboratory Animal Unit of the University of Hong Kong. The criteria outline in the “Guide for the Care and Use of Laboratory Animals” (NIH publication 86-23, 1985) were followed.

Bacterial Strain and Plasmid

Salmonella typhimurium aroA strain SL 7207 (S. ty) was kindly provided by Dr. D. Stoker and used as the carrier of the oral DNA vaccine in the study. DNA-HBsAg (pRc/CMV-HBs) was a generous gift from Dr. R. Whalen and was used for the transformation of S. ty (oral DNA vaccine) and for intramuscular immunization (DNA vaccine).

Schedule of Immunization and Evaluation

Twenty-eight HBs-tg mice were numbered and randomly divided into 5 groups (5 to 6 mice per group). Two groups were anaesthetized with an identical dose of sodium barbital and injected intramuscularly (tibialis anterior muscle of both hind legs) with 2 μg per dose of commerical protein HBsAg vaccine (HB-VAX II, MSD, USA) (protein vaccine) or 100 μg DNA vaccine per dose for four times at 3-weekly intervals. Two other groups were orally given one dose of 6×10⁹ colony forming units of either oral DNA vaccine or only the bacterial carrier S. ty per mouse. The fifth group of five unimmunized mice served as controls. Serum samples were collected from mice before and after immunization at 3-weekly intervals to monitor levels of HBsAg, anti-HBs and alanine aminotransferase (ALT). The mice were sacrificed on week 12. The spleen, liver and kidneys were taken for evaluation of cellular immune responses, transgenic DNA and mRNA, and immunohistopathological changes in liver and kidney tissues after immunization.

Since it was found that the oral DNA vaccine evoked an early immune response associated with a transient increase in serum ALT level at week 3, another set of twenty-four HBs-tg mice were recruited for the further study. They were randomly divided into 2 groups. One group was immunized with one dose of oral DNA vaccine and another with bacterial carrier control. Serum samples were obtained weekly for 4 weeks to measure levels of antibody and ALT. Three mice from each group were sacrificed at week 1, 2, 3 and 4 respectively. The spleen, liver and kidneys were collected to examine for the early inflammatory response. Twelve each of non-tg C57/6J and Balb/c mice were also vaccinated with either oral DNA vaccine or its blank bacterial control. Sera taken weekly from these animals were tested for ALT levels in the first 3 weeks to determine if the liver damage might be caused by bacterial toxic effect.

Serological and Biochemical Analysis

Serum HBsAg and anti-HBs were assayed by ELISA (BIOKIT, S.A. Spain) and quantified using a panel of HBsAg calibrators (Abbott Diagnostics, Chicago, USA) and standard positive controls of anti-HBs provided with the kit. The subtypes of these antibodies were identified by ELISA using the same kit, substituted with HRP-conjugated sheep anti-mouse IgG, IgG1 and IgG2 (SeroTec, UK) respectively. Hepatocellular injury was monitored by measuring serum ALT levels using a multiple-point rate colorimetric method with the Vitros 950 dry-chemistry analyzer (Ortho Clinical Diagnostics, Inc., Rochester, N.Y., USA).

Detection of T Cell Mediated Responses Post-Vaccination

Splenocytes from individual mice were suspended in 10% FCS-RPMI 1640 at a concentration of 5×10⁶ per ml and stimulated with 10 μg/ml of purified HBsAg (Aldevron, Fargo, N. Dak., USA) for three days. The supernatant of the cultures were collected at 24, 48 and 72 hours post stimulation for interferon IFN-γ_{.} and interleukin IL-4 assay respectively by ELISA using OptEIA kits (PharMingen, USA).

Cells were further cultured in the presence of 25 IU/ml of murine rIL-2 (R&D Systems, USA) for an additional 4-5 days. The CTL activity of the splenocytes was measured in triplicates using a standard four-hour calcein release assay in U-bottom 96-well microplates. Targets used in CTL assays were the splenocytes of normal C57/6J mice infected for 12 hours with 10 PFU/cells of Vaccinia-HBsAg virus (Vac-HBsAg) or blank Vaccinia virus (Vac-blank) as negative controls, both being generous gifts from Dr. Y. Wong. The cytolysis of the targets was determined by measuring the fluorescence intensity (FI). Percentages of specific cytolysis were calculated as follows:

$$( 1 - \frac{Experimental FI - Totall ysis FI}{Total control FI - Totall ysis FI} ) \times 100 \%$$

Semiquantification of Transgene DNA and mRNA by PCR and RT-PCR

The DNA and mRNA were separately extracted from mouse liver tissues using QIAamp DNA mini kit (Qiagen, USA) and mRNA Isolation Kit (Roche Molecular Biochemicals, Germany). The first strand cDNA was synthesized using RNA H+ Reverse Transcriptase (Life Technologies, USA) followed by one cycle of PCR. Quantification of the HBsAg transgene DNA and the transgene-encoded mRNA was performed by LightCycler PCR (LC-PCR) using a set of inner primers for the HBsAg region (forward, 5′-AAC ATG GAG AAC ATC ACA TC-3′; and reverse, 5′-AGC GAT AAC CAG GAC AAG TT-3′), which yielded a 203 bp product. A donor fluorescein probe (5′-ATT GAG AGA AGT CCA CCA CGA GAC TAG AC-fluorescein-3′) and acceptor LightCycler-Red 640 probe (5′-LC-Red 640-CTG TGG TAT TGT GAG GAT TCT TGT CAA CAA G-3′) directed to the 203 bp product were designed for the assay. LC-PCR was carried out using the LightCycler-FastStart DNA Master Hybridization Probes Kit (Roche Molecular Biochemicals, Germany) and LightCycler (Roche Diagnostics, Mannheim, Germany) according to the manufacturer. A ten-fold serial dilution ranging from 0.015 pg/ml to 150 pg/ml of calibrator 5 from the Hybrid Capture II (HCII) assay (Digene Corp, Beltsville, Md., USA) was employed as quantification controls.

Immunohistopathological Study

The liver and kidney portions were immediately either frozen in liquid nitrogen or fixed in 10% buffered formaldehyde and embedded in paraffin. Sections were made at 4 or 6 μm thickness and mounted on slides. Histopathological changes were examined by haematoxylin and eosin (H&E) staining, while viral gene expression in liver tissues were examined by immunohistochemical staining using goat anti-HBsAg, rabbit anti-goat biotinylated antibody and streptavidin_HRP-conjugate (DAKO, USA) according to standard procedures.

Statistical Analysis

The significance of differences between groups was analyzed by the paired Student's T-test.

Results

Oral DNA Vaccination Arrested Serum HBsAg Accumulation

The serum HBsAg levels in samples obtained at week 0 were essentially similar for all five groups of HBs-Tg mice (p>0.30). In the bacterial carrier and unimmunized groups, serum antigen levels continually increased over the 12-week period of observation, from the mean value of 60±9 ng/ml to 162±10 ng/ml and 59±14 ng/ml to 176±15 ng/ml at week 12, respectively. Evidently, the rate of secretion of the expressed antigen into peripheral blood exceeded clearance, so that there is an increasing accumulation of the antigen in the blood during the course of the experiment. In the animals immunized intramuscularly with the protein or DNA vaccine, the serum antigen levels increased from 60+5 ng/ml to 130+35 ng/ml and 58±7 ng/ml to 114±29 ng/ml respectively at week 3. Thereafter, the serum antigen level in the protein vaccine immunized group remained unchanged, while that in the DNA vaccinated group declined slightly to 94±6 ng/ml at week 12. In contrast, in the animals given a single dose of oral DNA vaccination, serum antigen accumulation was arrested at as early as week 3 and the serum HBsAg level was significantly lower than those in the other groups throughout the duration of the twelve weeks (p>0.01).

Oral DNA Vaccination Evoked a Rapid Specific Antibody Response

In the HBs-tg mice given a single dose of oral DNA vaccine, serum anti-HBs increased rapidly, reaching 45±8 mIU/ml at week 2 (FIG. 12), and thereafter slightly increased to 74±20 mIU/ml at week 12 (FIG. 12). This was concomitant with arrest of HBsAg accumulation in these animals. Conversely, the animals primed and boosted intramuscularly with 3 doses of protein or DNA vaccine did not develop significant antibody responses to HBsAg until after week 9. An additional boost dose administered to these animals triggered specific antibody response, respectively reaching levels of 36±26 and 167±53 mIU/ml at week 12. All serum samples from the 2 groups of control mice were consistently negative for anti-HBs throughout the course of the experiment.

Oral DNA Vaccine Triggered at Vigorous Th 1 and CTL Response

Serum samples taken at week 12 were further tested for contents of total IgG, IgG1 and IgG2 subtypes of the viral antibody (FIG. 12). In the oral DNA vaccinated animals, the induced antibody was mainly subtype IgG2. Intramuscular protein immunization primed IgG1 subtype antibody response and, the specific antibody response to intramuscular DNA vaccination was dominated by subtype IgG1, which was accompanied by detectable amounts of subtype IgG2.

Splenocytes obtained from the immunized and control animals were stimulated with purified HBsAg and the activation of Th 1 and Th 2 cells was respectively detected by IFN— and IL-4 induction assays. Splenocytes obtained from oral DNA vaccinated mice at week 12 produced the highest level of HBsAg-induced IFN-γ, reaching 2801±480 pg/ml at 72 hours post-stimulation. This was significantly higher than those in the groups immunized intramuscularly with the DNA vaccine (2044±639 pg/ml at 72 hours; p=0.03) and the protein vaccine (607±639 pg/ml at 72 hours; p<0.01). No significant HBsAg-induced IFN-γ_{.} was detected from the splenocyte cultures of control animals. Unlike the secretion of IFN-γ_{.}, no significant increase of IL-4 production was observed in the animals immunized with oral DNA vaccine, and only low levels of IL-4 were exhibited in the cultures from the mice immunized intramuscularly with protein and DNA vaccines (data not shown). Secretion of IFN-γ_{.} was detectable in 72-hour cultures of splenocytes taken from HBs-tg mice at week 1 post-immunization with the oral DNA vaccine. Levels of this cytokine increased quickly thereafter, reaching levels of 1986±164 pg/ml at week 2, 2636±335 pg/ml at week 3 and 2786±513 at week 4. IFN-γ_{.} elaboration was much lower in 72-hour cultures of spleen cells from the mice immunized by bacterial carrier, at 192±49, 420±64, 268±65 and 267±27 pg/ml respectively at week 1 to 4.

In the animals immunized with the oral DNA vaccine or given intramuscularly the DNA vaccine, mouse spleen cells were vigorously cytotoxic against Vac-HBsAg infected target cells, but did not exhibit cytotoxicity against the viral control targets. CTL response was barely induced in protein HBsAg immunized mice and the control animals. Importantly, vigorous antigen-specific cytoctoxicity was detected in splenocyte cultures of the mice as early as 2 weeks after they received oral DNA vaccination, although it was accompanied by a low level of non-specific cytolysis of Vac-blank infected control target. Spleen cell cultures from the mice given the bacterial carrier also showed low levels of non-specific cytolysis against both Vac-HBsAg and Vac-blank targets, suggesting that infection by the bacterial carrier itself may induce non-specific innate immune response.

In summary, the oral DNA vaccine evoked a significant Th 1-type response, which was characterized by IgG2 subtype antibody response, early and vigorous antigen-induced IFN-, and strong CTL activity. This specific cellular response may have been preceded by an innate immune response caused by bacterial carrier infection. The protein vaccine elicited a weak Th 2 type response, but the animals did not show significant Th 1 and CTL responses, while intramuscular DNA immunization evoked IgG1 antibody, Th 1 & 2, and CTL responses.

Oral DNA Immunization Down-Regulated the Transcription and Expression of HBsAg-Transgene in Liver Tissues

Liver tissues from six mice given the oral DNA vaccine showed fewer HBsAg positive hepatocytes (FIG. 7A) when compared with those given bacterial carrier (FIG. 7B) and the other vaccinations (data not shown), which were uniformly stained for HBsAg. The extent of reduction in expression of HBsAg varied among the animals and was most pronounced in two mice, in which HBsAg staining was negative in patchy areas of the liver sections. DNA and mRNA were extracted from liver tissues for determination of amounts of HBsAg transgene and levels of transcription (Table). The transgene DNA contents were the same for different groups of animals (p>0.43). The levels of the viral transcript mRNA were essentially the same for the control mice and those which received the DNA protein vaccines. However, the oral DNA vaccine was found to reduce the level of the viral-transcript by at least 4 fold (p<0.02). The results thus suggest that the suppression of HBsAg expression in hepatocytes by the oral DNA vaccine is most likely due to down-regulation of transcription of HBsAg-mRNA in the liver tissues.

Oral DNA Vaccination Elicited a Transient Inflammatory Response and Liver Injury at the Early Stage of Immunization

It was notable that oral DNA vaccination caused an intense inflammatory response, resulting in one death (1/15) due to fulminant hepatitis 13 days after vaccination. The diagnosis of severe hepatitis was confirmed by characteristic liver pathology showing intense lymphocytic infiltration. Focal aggregation of mononuclear inflammatory cells, mostly lymphocytes, and vacuolar degeneration of scattered liver cells, were seen in the liver section (FIGS. 8A, 3-F). Mild and focal lymphocytic infiltration was observed in liver tissues taken from mice of groups 3 and 4 as early as week 1 (FIGS. 9, A-3a and A-4e). Lymphocytic infiltration became most intense in samples of group 3 at week 2 with marked eosinophilic degeneration of hepatocytes (A-3b), but declined thereafter (A-3c and A-3d). Liver tissues of group 4 also showed intense lymphocytic infiltration at week 2 (A-4f) but it was milder than that of group 3 with the hepatitic activity reduced and mitotic liver cells appeared in subsequent samples (A-4 g & -4 h). Serum ALT levels were markedly raised in the surviving mice of group 3 at week 3 post-immunization. They were about 14 fold higher than those of pre-vaccination (p<0.001), but the increase was transient and the ALT level returned to normal in subsequent samples (FIG. 10A). Control animals given the bacterial carrier also showed a moderate increase in the ALT level at week 3, but the level was significantly lower than that of oral DNA vaccinated mice (p<0.001). This liver injury was also transient and the ALT level returned to normal thereafter. A detailed study in the first 4 weeks of vaccination showed that serum ALT levels in the oral DNA vaccinated mice increased at week 1, reaching the highest level of 2203±153 U/ml and declined thereafter to an almost normal level at week 4 (FIG. 10B). This was unlikely to be related to the bacterial toxic effects because the ALT levels of non-transgenic C57/6J and Balb/c mice did not change after receipt of oral DNA vaccine and its bacterial carrier (data not shown). The animals given the protein or DNA vaccine, and the unimmunized mice, had normal ALT levels throughout the duration of the experiment. Moreover, no significant histopathological changes in the liver tissues from all groups of animals were observed at the end of immunization, including the animals immunized with the oral DNA vaccine (FIGS. 8A, 3A to 3E) and the bacterial carrier (FIGS. 8B, 4A to 4E).

Oral DNA Vaccination Induced Cytopathic and Non-Cytopathic Suppression of HBsAg-Transgene in Early Stage

Oral DNA vaccination triggered an early inhibition of transgene expression in the liver tissue by both cytopathic and non-cytopathic pathways (FIG. 9B). The liver cells showed diffuse HBsAg immunoreactivity. The apparent decrease in immunoreactive hepatocytes was found in liver sections taken from the mice at week 2 (FIGS. 9B-3j) and the mouse dying on day 13 after receiving oral DNA immunization (FIGS. 7A, 3F). The livers showed heavy lymphocytic infiltration, scant region due to cytolysis of hepatocytes, eosinophilic degeneration of HBsAg-positive hepatocytes and few antigen-negative normal liver cells. In the liver samples of week 3 and 4, viral antigen expression was still curtailed markedly and lymphocytic infiltration declined, featuring an increase of HBsAg-positive necrotic and HBsAg-negative normal liver cells accompanied by a reduction in eosinophilic degeneration of hepatocytes. There was no significant difference between HBsAg-expression in liver tissues of groups 3 and 4 at week 1 (FIGS. 9B-3i and -4m). However, animals vaccinated with the bacterial carrier seemed to experience a transient cytolytic suppression of the transgene as shown in FIGS. 9B-4n but did not exhibit non-cytolytic inhibition in the subsequent samples at weeks 3 and 4 (FIGS. 9B-4o and -4p). Since splenocytes taken from oral DNA vaccinated mice at weeks 2 to 4 after immunization showed both specific and non-specific cytotoxicity while those from bacterial carrier immunized mice exhibited non-specific cytolytic activity only, the results thus suggest that the inhibitory effects in the early stage were brought about initially by a cytolytic pathway and switched later to a non-cytolytic pathway in the former but only a non-specific cytolytic pathway in the latter.

Discussion

In this study, we have compared the therapeutic effect of three vaccines of different formulation and administrated by different routes on the state of HBsAg immune tolerance in HBs-tg mice. Our results showed that although different vaccinations and formulations reversed the state of immune anergy prevailing in HBs-tg mice with essentially of a quantitative nature, only oral DNA vaccine suppressed expression of the viral transgene.

In agreement with previous studies, our study showed that three doses of intramuscular immunization with the protein or DNA vaccine did not induce significant immune responses to HBsAg in the HBs-tg mice. With the protein vaccine, reversal of immune energy was only evidenced after week 9 post-vaccination by the detection of a low level of antibody dominated by the IgG1 subtype. The animals also exhibited a weak Th 2 response, but they did not show detectable specific CTL or Th 1 activity. The intramuscular DNA vaccine evoked a vigorous antibody response mainly of IgG1 subtype and Th and CTL responses by the third booster dose. Antigen accumulation was arrested after the second dose of the DNA vaccine and the third dose of the protein vaccine. However, immunity thus acquired by either group of animals was inadequate to control the expression of the viral gene. The level of the transgenic mRNA detected in the liver tissues and the amount of liver cells expressing the viral antigen in these mice were essentially the same as the control animals. The arrest of viral antigen accumulation in serum samples may be explained by the more efficient clearance of the antigen from the peripheral blood in the forming of immune complexes with the newly produced antibody. It is also possible that the antigen is less readily detectable as immune complex than as free antigen. The results indicated that repeated doses of these two vaccines were necessary for reversal of HBsAg immune energy. Booster effects seen with successive doses were probably due to increasing inflammatory reactions and recruitment of APC to sites of inoculation. Our result is different from the study of Mancini et al., which showed that one dose of intramuscular DNA vaccination induced the clearance of serum HBsAg and down-regulation of transgene expression in liver tissue in HBV-Tg mice. This may be due to the use of different Tg mouse lineage, E36, in their experiment but 44Bri in our study.

A single dose of oral DNA vaccination evoked a Th 1-type response featuring a vigorous response by the CTL and Th 1 subset of immune T cells, and Th 1 dependent production of IgG2 subtype antibody, leading to a decline of serum HBsAg in the HBs-tg mice. Importantly, the transcription and expression of the HBsAg gene in the host hepatocytes were evidently suppressed by the vaccination. The efficacy of oral DNA vaccine compared with the others was attributed, at least in part, to the immune response being orchestrated by professional APC presented endogenous antigens. Our previous studies showed that immunization by the oral DNA vaccine brought about the process of an active intracellular infection in the intestinal tract. This was demonstrated by induction of IgG2 subtype antibodies against Salmonella and further confirmed by the heat-inactivated version of this oral DNA vaccine and E. coli which harbored the same plasmid DNA not eliciting the specific immune response. It appeared that the vaccine bacteria were effectively ingested by resident APC and by those, which were recruited to the site of infection. The bacterial carriers undergo autolysis due to the presence of a mutation (AroA) shortly after being engulfed by APC. The released DNA vaccine could enter the nucleus and the harbored gene could be efficiently expressed in APC. The resulting bacterial debris can act as an adjuvant to up-regulate the expressed antigen being presented on cell membrane together with MHC. Presentation of the endogenous antigen by APC, with concomitant activation by bacterial endotoxin, was found to evoke a Th 1-type response in mice featuring induction of IgG2 antibody, vigorous Th 1 cell and CTL response. The crucial role of APC in orchestrating the immune response to the vaccine was supported by the finding that infusion of activated peritoneal macrophages previously infected with the oral DNA vaccine triggered essentially the same pattern of immune responses as oral DNA vaccination. The presence of inflammatory cytokines secondary to the innate immune response in the micro-environment may further activate the resident and recruited APC, possibly by up-regulation of co-stimulatory molecules, e.g. MHC class II and B7, which deliver not only stimulatory but also survival signals to T cells. Furthermore, it was recently reported that S. typhimurium infection could stimulate DC to increase secretion of IL-12, which is the initial signal for triggering a specific immune response.

Our results showed that only the oral DNA vaccine can inhibit expression of the transgene in liver tissues. Previous studies by other investigators showed that protein-based and DNA-based vaccines elicited specific humoral and cellular immune responses, leading to clearance of serum viral antigens, but could not suppress viral gene expression in hepatocytes. None of the therapeutic vaccine candidates were able to evoke a hepatitic flare. Furthermore, adoptive transfer of cytokine-activated DC or specific T cells was also unable to abolish transgene expression nor display infiltration of specific T cells in the liver of HBs-tg mice. The most distinguishing feature of our approach is that the oral DNA vaccine initiates an active intracellular infection of Salmonella which activates the innate immune system including NK and NKT cells. Activated NK cells may restore their effector functions, i.e., causing cytolysis of target cells and producing inflammatory cytokines, thus resulting in mild inflammation and tissue damage in the liver of HBs-Tg mice. This is consistent with our observation that bacterial carrier vaccination also led to a slight and transient non-specific cellular response and liver injury in HBs-Tg mice but not in normal mice. Subsequently, the innate immune response probably triggered an undefined change of the microenvironement that enabled the adaptive CTLs to infiltrate the liver and effect the target cells, as in previous reports of the abolishment of expression of viral genes in the liver resulting from unrelated intracellular infection.

The suppression of transgene in the liver tissues of the oral DNA vaccine immunized mice is achieved by both cytopathic and non-cytopathic pathways in the early stage. This was evidenced by hepatic immune-histopathological study of the mice in the first 4 weeks. The cyolytic reaction was most pronounced at week 2. Liver sections obtained at the time, including that from a mouse which died on day 13, exhibited intense cytolytic scanty and eosinophilic degeneration of liver cells with only few normal cells. It is possible that inflammatory cytokines induced by innate immune response, such as IL-2 and IFN-_{.}γ, may stimulate MHC class I and proteasome subunit expression in hepatocytes, thus enhance the viral epitope processing and presentation in these target cells. This may favor the cytolytic effect directed by specific CTLs. Whatever the explanation, the ctolytic activity subsided by week 4, with an increase of necrotic HBsAg positive liver cells and apparent normal HBsAg negative hepatocytes in subsequent samples. The transgene suppression was likely to be sustained thereafter by a non-cytolytic pathway only, because no detectable liver injury was shown as determined by serum ALT levels and the histopatholgy of liver sections in these mice. Homeostasis regulating the switch from cytolytic to non-cytolytic mechanisms remains to be further studied, but could be ascribed to increasing production of antiviral cytokines. It had been shown that both CTLs and cytokines could inhibit viral gene expression and virus replication without destruction of those infected cells in HBV-Tg mice. This could be attributed to antiviral cytokines, especially IFN_{.}-γ and TNF-_{.}α, in chimpanzees with acute experimental HBV infection. Unrelated intracellular infections may also induce noncytolytic anti-HBV effects via the induction of antiviral cytokines. The non-cytopathic suppression of HBsAg transgene observed in the oral DNA vaccinated mice may possibly be mediated via the same mechanism, e.g. anti-viral IFN-γ_{.}, which appeared earlier and was more vigorous in splenocyte cultures of this group than those of other groups after stimulation with HBsAg.

It was notable that the oral DNA vaccine evoked an intense inflammatory response in the early stage, which resulted in one animal dying of acute hepatitis 13 days after vaccination. Serum ALT levels of the surviving mice were markedly raised in samples taken at week 2 after immunization but returned to normal in subsequent samples taken at week 4 and after. Since almost all liver cells of HBs-Tg mice express the transgene, survival of these mice (14/15) from acute hepatitis may probably be attributed to the dominance of non-cytolytic over cytolytic effects in these animals. The non-cytolytic effect inhibiting the downregulating viral gene expression can prevent excessive cytolysis of target hepatocytes mediated by CTL. However, the underlying mechanism requires further investigation by more intensive temporal studies. The clinical significance of a hepatitic flare possibly caused by the oral DNA vaccination in human remains to be elucidated. In this regard, unlike HBV-Tg mice, only a fraction of liver cells are infected by HBV in human. Furthermore, virus replication and viral antigen expression in infected hepatocytes can be minimized by a preceding course of antiviral treatment, which would be helpful to alleviate the liver injury associated with the vaccination. Finally, it was of interest to note that liver injury associated with the oral DNA vaccine appears to parallel findings in cases of successful clearance of HBV in HBsAg positive bone marrow transplant recipients who also experience an acute hepatitis episode following adoptive immune transfer from HBV immune donors.

HBV transgenic mice are the only animal models available for studying immune tolerance prevailing in chronic HBV infection. Although not directly applicable, it is hoped, nevertheless, that findings can provide a guide as to the feasibility of immune intervention in chronic human HBV infection. In the present study, we have shown that a single dose of the oral DNA vaccine not only triggers vigorous cellular response but also suppresses expression of the viral gene in the animal model. Additional studies are required to further understand the mechanism underlying this strategy of therapeutic immunization in different transgenic mice models, especially in those with active virus replication. However, the present and previous findings taken together are consistent with the notion that the oral DNA vaccination may be a feasible approach to immune intervention of chronic HBV infection.

### Example 5

### Unique Immunogenicity of Hepatitis B Virus DNA Vaccine Presented by Live-Attenuated Salmonella typhimurium

A novel vaccine for hepatitis B virus (HBV) was designed by putting a naked DNA vaccine carrying hepatitis B surface antigen (HBsAg) into live-attenuated Salmonella typhimurium. Mucosal immunization by the oral route in mice showed significantly stronger cytotoxic T lymphocyte (CTL) response than recombinant HBsAg vaccination (P<0.01 at an effector:target ratio of 100:1), while comparable to intramuscular naked DNA immunization at all effector:target ratios. Contrary to previous reports on naked DNA vaccines given intramuscularly, the IgG antibody response induced by the mucosal DNA vaccine is relatively weak when compared to recombinant HBsAg vaccine (P<0.001 at day 21). These findings are supported by a high interferon-γ_{.} but a low interleukin-4 level detected in the supernatant of splenic cell cultures obtained from mucosally immunized mice. As distinct to recombinant HBsAg vaccine which is effective for protection, oral mucosal DNA vaccine should be considered as a candidate for therapeutic immunization in chronic HBV infection, donor immunization before adoptive transfer of HBV-specific CTL to HBsAg positive bone marrow transplant recipients, and immunization of non-responders to recombinant HBsAg vaccine. This strongly cellular and relatively absent humoral response may make this vaccine a better candidate as a therapeutic vaccine for chronic HBV carriers than naked DNA vaccines, as the humoral response is relatively less important for the clearance of HBV from hepatocytes, but its presence may lead to side effects such as serum sickness and immune complex deposition in chronic HBV carriers.

Materials and Methods

Animals

Female Balb/c (H-2^{d}) mice (6-8 weeks old, 18-22 g) were used in all the animal experiments. They were housed in cages, under standard conditions with regulated day length, temperature and humidity, and were given pelleted food and tap water ad libitum.

Transfection of 293 Cells with pRc/CMV-HBs(S)

Two hundred and ninety three cells were plated at 1×10⁷ cells per wall in DMEM (Gibco-BRL) with 10% foetal calf serum (FCS) in a 6-well plate on the day before transfection. On the day of transfection, each well was transfected with 1 μg plasmid encoding eukaryotically expressed HBsAg [pRc/CMV-HBs(S)], a gift from Dr. Robert Whalen, or distilled water (negative control) with FuGENE 6 Reagent (Boehringer Mannhein, Germany) according to manufacturer's instructions. 48 hours after transfection, the cells were harvested and lysed by freezing and thawing 3 times. After centrifugation at 14 000 rpm, the supernatant was used for the measurement of HBsAg.

In vitro Infection of Macrophages with Live-Attenuated S. Typhimurium Transformed with pRc/CMV-HBs(S)

Hundred microliter of Conconavalin A (Sigma) in 1 ml serum-freeRPMI (Gibco-BRL) was injected intraperitoneally to 2 Balb/c mice. The mice were euthanised after three days. Primary peritoneal macrophages were harvested by washing the peritoneal cavities of the mice with 10 ml serum-free RPMI. After washing twice, the macrophages were pooled and resuspended at 5×10⁶ cells/ml with serum-free RPMI. The macrophages were incubated in 6-well plates at 2×10⁷ cells per well at 37° C. for two hours. After removing the non-adherent cells and washing once with serum-free RPMI, the macrophages were infected with auxotrophic S. typhimurium aroA strain SL7207 (S. Typhimurium 2337-65 derivative hisG46, DEL 407 [aroA:Tn10{Tc-s}]), a gift from Dr. Bruce Stocker transformed with pRc/CMV-HBs(S) or auxotrophic S. typhimurium aroA strain SL7207 (negative control) at MOI 10. The cultures were further incubated at 37° C. for 30 min. After washing twice, the remaining extracellular bacteria were killed by addition of gentamicin in RPMI (50 μg/ml) supplemented with 10% FCS. After incubation for 4 h at 37° C., the intracellular bacterial multiplication was inhibited by addition of tetracycline (10 μg/ml). The cultures were incubated at 37° C. Cells were harvested at 24, 48 and 72 h and lysed by freezing and thawing 3 times. After centrifugation at 14 000 rpm, the supernatants were pooled and used for the measurement of HBsAg.

Measurement of Hepatitis B Surface Antigen

Hundred microliter of each cell lysate was added to an ELISA plate precoated with guinea pig anti-HBsAg antibodies (Biokit, Spain). The plate was incubated at 37° C. for 1 h. After washing with washing solution 3 times, 100 μl peroxidase-conjugated goat anti-HBsAg antibody diluted according to manufacturer's instructions was added to the wells and incubated at 37° C. for 30 min. After washing with washing solution 3 times, 100 μl diluted 3,3′, 5,5′-tetramethylbenzidine (TMB) was added to each well and incubated at room temperature (RT) for 30 min. 100 μl of 1 M H₂SO₄ was added and the absorbance of each well was measured at 450 nm, using TMB buffer as a blank. Each sample was tested in duplicate and the mean absorbance for each serum was calculated.

Immunization Schedule

Twenty four Balb/c mice were used for the immunization experiments. On day 0, 6 mice of each group were immunized intramuscularly (tibialis anterior muscle) with pRc/CMV-HBs(S) (100 μg per mouse, DNA vaccine group), orally with auxotrophic S. typhimurium aroA strain SL7207 transformed with pRc/CMV-HBs(S) (6×10⁹ bacterial cells per mouse, mucosa DNA vaccine group), intraperitoneally with HBsAg vaccine with alum adjuvant (H-B-VAX II, MSD, 0.5 μg per mouse, proteing vaccine group), or orally with S. typhimurium aroA strain 6×10⁹ bacterial cells per mouse, control group), respectively.

Measurement of Serum Antibodies Against HBsAg

Mice from each group were bled on days −1, 7, and 21. The blood was centrifuged at 2700×g for 20 min and the supernatant (serum) was stored at −70° C. before antibody measurement. Mouse sera (diluted with PBS-2% BSA) were added to ELISA plates precoated with HBs Ag (Biokit, Spain). The plates were incubated at 37° C. for 1 h. After washing with washing buffer 3 times, 100 μl peroxidase-conjugated goat anti-mouse antibody (Zymed Laboratories Inc.) diluted according to manufacturer's instructions using PBS-2% BSA were added to the wells and incubated at 37° C. for 30 min. IgM and total IgG levels were assayed to assess the primary and secondary immune response, while IgG1 and IgG2a were used to determine whether the humoral response was inclined towards the Th 2 or Th 1 pattern, respectively. After washing with washing buffer 3 times, 100 μl orthophenylenediamine (OPD) substrate (prepared by diluting 2 mg OPD [Calbiochem] in 2.5 ml 50 mM citric acid [pH 5] with 2.5 μl 30% H₂O₂) was added to each well and incubated at RT for 30 min. Hundred microliter of 1 M H₂SO₄ was added and the absorbance of each well was measured at 492 nm, using OPD buffer as a blank. Each sample was tested in duplicate and the mean absorbance for each serum was calculated. The serum antibody level of a particular mouse on a particular day was defined as the absorbance obtained from the serum on that day minus that of the corresponding mouse on day −1.

Cytotoxic T Lymphocyte Assay

P815 cells stably expressing HBsAg (P815-HBsAg) were a gift from Dr. Jorg Reimann. Cytotoxic T lymphocyte (CTL) activity was assayed in triplicate in a standard 4 hour calcein AM release assay. Spleen cells harvested from immunized mice on day 25 were stimulated in vitro with_{.} γ-irradiated P815-HBsAg cells at a spleen cell/stimulator ratio of 20:1 for 3 days. Murine recombinant interleukin 2 (rIL-2) (25 IU/ml) was then added and the culture was incubated for another 4 days. The resultant stimulated and expanded spleen cells were purified by Ficoll-Hypaquet (Pharmacia Biotech, Sweden) and were used as the effector cells. Target cells (P815-HBsAg and P815 cells were labeled immediately before use by incubating the cells in a predetermined optimal concentration of Calcein AM (2 μM) at 37° C. for 40 min, washed, and resuspended at 5×10⁴ cells per ml. Target cells (100 μl) were incubated with an equal volume of effector cells in 96-well U-bottomed microtitre plates at effector:target (E:T) ratios ranging from 0.3:1 to 100:1. The plates were centrifuged at low speed for 3 min and incubated at 37° C. for 4 h. The cytolysis of targets was determined by measuring calcein AM fluorescence using a fluorometer. The maximum release was estimated by incubating target cells with 5% SDS (Sigma) (total) and spontaneous release estimated by incubating the targets in medium alone (control). The percentage specific target lysis is calculated by the following formula:

1-(fluorescenceₛₐₘₚₗₑ−fluorescenceₜₒₜₐₗ)/(fluorescence_{control}−fluorescenceₜₒₜₐₗ)×100%

Interleukin and Interferon γ-Assays

During the stimulation of splenic cells harvested from immunized mice by γ-irradiated P815-HBsAg, 200 μl of supernatant from each sample was collected at 24, 48, and 72 h for cytokine measurement. Monoclonal antibodies against IL-4 or IFN-γ_{.} were coated onto wells in 96-well microtitre plates (OptEIA, PharMingen, Becton Dickinson) at 1:250 dilutions according to manufacturer's instructions. The plates were incubated at RT for 24 h. After washing with washing buffer 3 times, the plates were blocked with assay diluent at RT for 1 h. After washing with washing buffer 3 times, 100 μl of supernatant from each sample was added to the wells in duplicate. The plates were incubated at RT for 2 h. After washing with washing buffer 5 times, 100 μl diluted biotinylated antibody against IL-4 or IFN-γ_{.} and avidin-horseradish peroxidase conjugate were added to the wells and incubated at RT for 1 h. After washing with washing buffer 8 times, 100 μl 3.3°-5,5′-tetramethylbenzidine substrate was added to each well and incubated at RT for 30 min. Hundred microliter of 0.3 M H₂SO₄ was added and the absorbance of each well was measured at 450 nm.

Statistical Analysis

Comparison was made among the serum antibody subtype levels at days 7 and 21, the percentage specific lysis of target cells at various E:T ratios, and the IL-4 and IFN-γ_{.} levels of supernatant at 24, 48, and 72 h for the 4 groups of mice using one-way ANOVA. P<0.05 was regarded as statistically significant.

Results

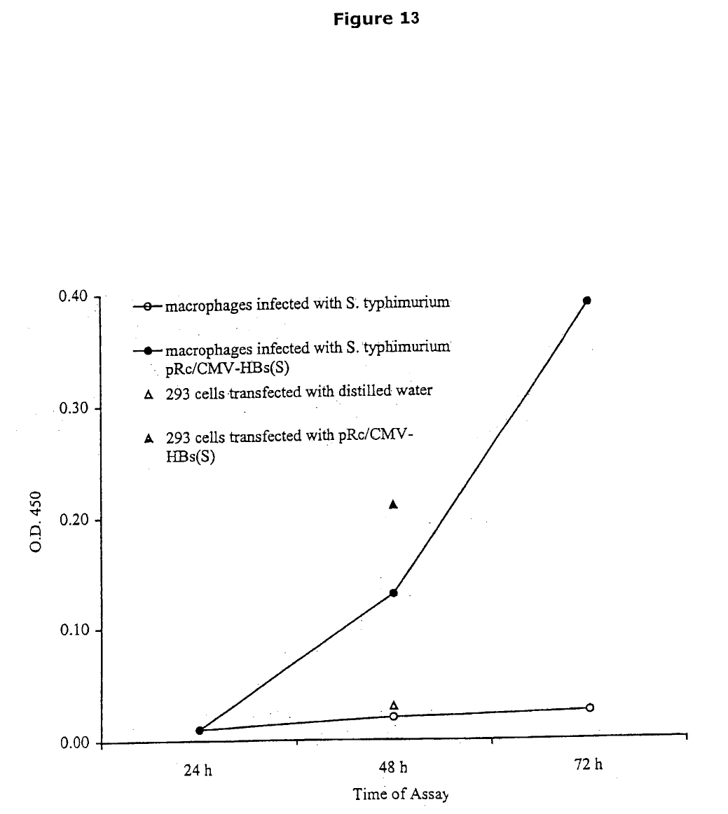

HBsAg Expression in 293 Cells Transfected with pRc/CMV-HBs(S) and Macrophages Infected with Live-Attenuated S. typhimurium Transformed with pRc/CMV-HBs(S) The HBsAg levels in the lysates of 293 cells transfected with pRc/CMV-HBs(S) harvested at 48 h post-transfection and macrophages infected with S. typhimurium pRc/CMV-HBs(S) harvested at 24, 48, and 72 h post-infection are shown in FIG. 13. Two hundred and ninety three cells transected with pRc/CMV-HBs(S) showed good expression of HBsAg at 48 h post-transfection, and macrophages infected with S. typhimurium pRc/CMV-HBs(S) showed good expression of HBsAg at 48 and 72 h post-infection.

Antibody Response

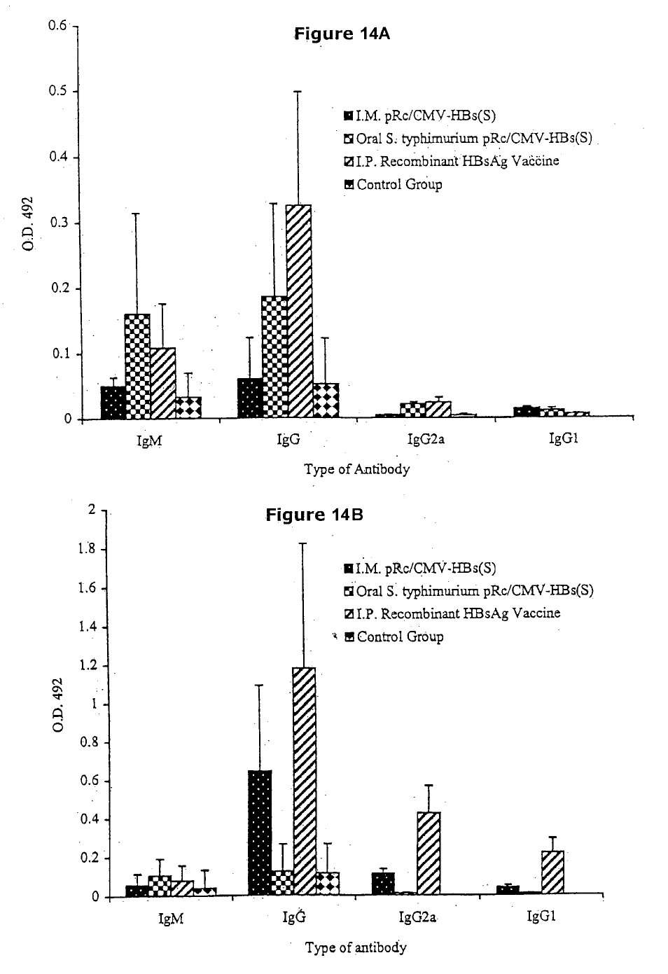

The antibody subtype levels of the 4 groups of mice on days 7 and 21 were summarized in FIGS. 14A and 14B, respectively. The serum IgG levels of the protein vaccine group were significantly higher than those of the control group at days 7 and 21, respectively (P<0.05 and <0.001). Moreover, the serum IgG levels of the protein vaccine group were significantly higher than that of the mucosal DNA vaccine group on day 21 (P<0.001). However, the OD values of IgG subtypes were too low for the evaluation of Th 1/Th 2 type response.

Cytotoxic T Lymphocyte Response

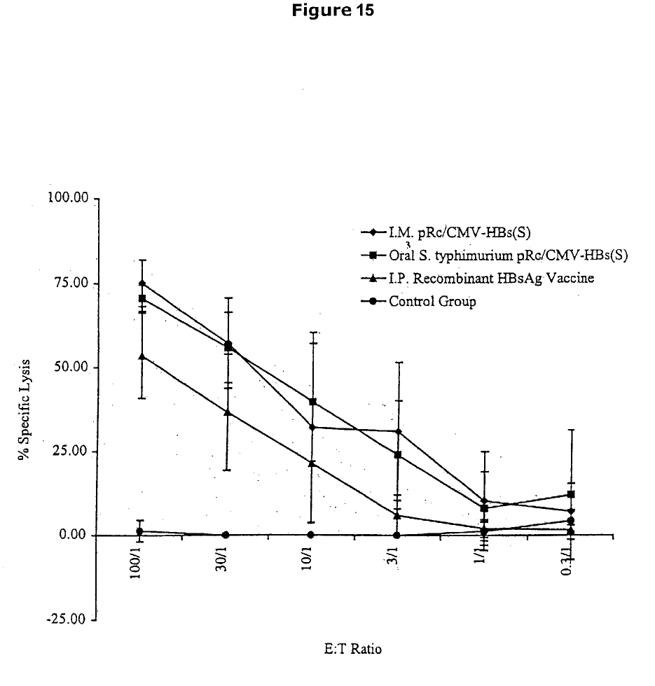

The percentage specific lysis of target cells at various E:T ratios in the 4 groups of mice were shown in FIG. 15. Splenic cells of the DNA vaccine, mucosal DNA vaccine, and protein vaccine groups all demonstrated efficient target cell lysis at minimum E:T ratios of ≧10:1. The percentage specific lysis of target cells of the DNA vaccine group were significantly higher than that of the control group at E:T ratios of 3:1, 10:1, 30:1, and 100:1 (P<0.01, <0.05, <0.0001, and <0.0001). The percentage specific lysis of target cells of the mucosal DNA vaccine group were significantly higher than that of the control group at E:T ratios of 10:1, 30:1, and 100:1 (P<0.01, <0.0001, and <0.0001). The percentage specific lysis of target cells of the protein vaccine group were significantly higher than that of the control group at E:T ratios of 30:1 and 100:1 (P<0.0001 and <0.0001). Furthermore, the percentage specific lysis of target cells of the DNA vaccine group were significantly higher than that of the protein vaccine group at E:T ratios of 3:1, 10:1. and 100:1 (P<0.05, <0.05, and <0.001); and the percentage specific lysis of target cells of the mucosa vaccine group was significantly higher than that of the protein vaccine group at E:T ratio of 100:1 (P<0.01). There was no statistically significant difference between the percentage specific lysis of target cells of the DNA vaccine and mucosa vaccine groups at all E:T ratios.

Cytokine Assays

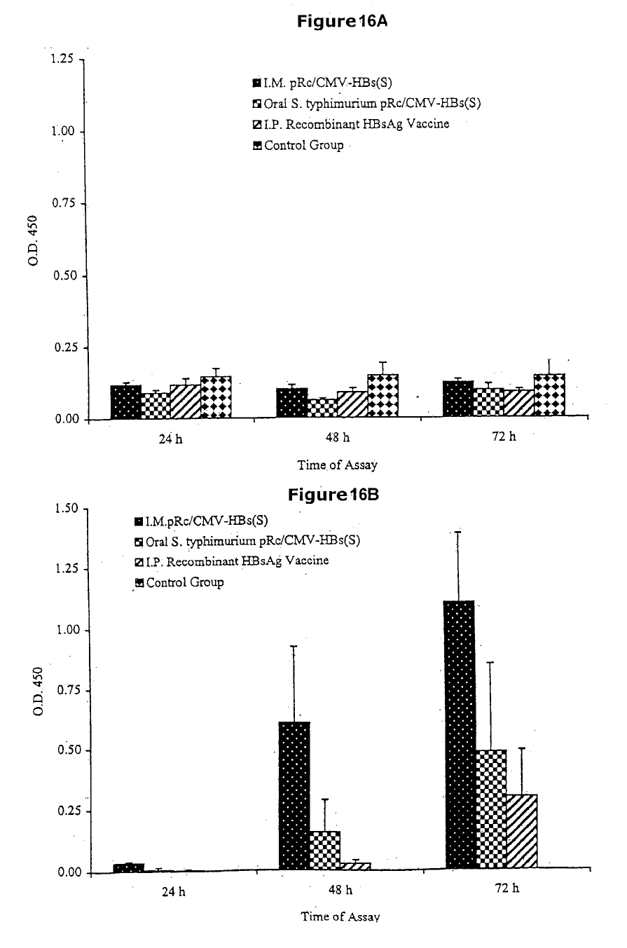

The IL-4 and IFN-γ_{.} levels of supernatant obtained at 24, 48, and 72 h from splenic cell cultures of the 4 groups of mice were shown in FIGS. 16A and 16B, respectively. The IFN_{.}-levels of the DNA vaccine group were significantly higher than those of the control group at 24, 48, and 72 h (P<0.0001, <0.05, and <0.005). Furthermore, the IFN-γ_{.} levels of the DNA vaccine group were significantly higher than those of the protein vaccine group at 24, 48, and 72 h (P<0.0001, <0.05, <0.05); and higher than that of the mucosal vaccine group at 24 h (P<0.001). The IL-4 levels of the 4 groups of mice were low at 24, 48, and 72 h; and there was no statistically significant difference in the IL-4 levels among the 4 groups of mice.

Discussion

Clinical trials in humans has demonstrated that the use of lamivudine, IFN-α_{.}, and recombinant HBsAg complexed with hepatitis B immunoglobulin (HBIG) can clear HBeAg in chronic HBV carriers in a certain proportion of cases. As for the clearance of HBsAg, only adoptive transfer of immunity from donors with natural immunity to HBV during bone marrow transplantation (BMT) has consistently demonstrated efficacy in our BMT recipients. Though one study in Caucasians succeeded in clearing HBsAg in chronic carriers by vaccination with recombinant HBsAg, such finding probably would not be reproduced in our population because our predominant mode of transmission of HBV is vertical. This was also reflected by the much lower efficacy of IFN-α_{.} in clearing HbeAg status in our previous studies. The failure of clearing HBsAg in HBV carriers by immunization with recombinant HBsAg is not unexpected because animal and human studies have consistently shown a strong antibody but a poor CTL response. Thus, the design of a vaccine or vaccine delivery system that can elicit a strong CTL response may be pivotal in achieving a therapeutic clearance of chronic HBV infection.

A unique strong CTL, but a relatively weak antibody response, was elicited by immunization with the live-attenuated S. typhimurium containing the DNA vaccine. It has been shown repeatedly, and was supported by the results of this study, that naked DNA vaccines are associated with stronger CTL response than recombinant protein vaccines. In the present study, we have also shown that a strong CTL response, comparable to that elicited by naked DNA vaccine and significantly better than that of recombinant protein vaccine, was associated with the administration of live-attenuated S. typhimurium containing the DNA vaccine. This strong CTL response was further supported by the detection of a high level of IFN-γ_{.}, a CTL-associated cytokine in the supernatant of the splenocyte cultures derived from mice immunized orally with live-attenuated S. typhimurium containing the DNA vaccine. On the other hand, the antibody response induced by live-attenuated S. typhimurium carrying the DNA vaccine is relatively weak. This is in contrast to the strong IgG response associated with both naked DNAand recombinant protein vaccines detected on day 21. This differential CTL/antibody inclined immune response induced by naked DNA, oral mucosal, and recombinant protein vaccines could be explained by the difference in the ways in presentation of the HBsAg antigen and the type of adjuvant. When mice are immunized with recombinant HBsAg, the main type of immune response generated is the antibody response, since the exogenous antigens is mainly presented by B cells through the MHC Class II pathway to Th 2 cells. In the case of naked DNA vaccine, part of the HBsAg generated in vivo in the muscle cells are secreted and presented by B cells through the MHC Class II pathway, leading to a good antibody response; while part of the antigen is cleaved within the antigen presenting cells and presented through the MHC Class I pathway, leading to a strong CTL response. On the other hand, it is difficult to understand why the same DNA, when carried by live-attenuated S. typhimurium, would induce a strong CTL but a relatively weak antibody response. We speculate that this may be due to the selective infection of mucosa associated lymphoid tissue (MALT) cells by the live-attenuated S. typhimurium. When the DNA encoding HBsAg was selectively carried into the MALT cells, most of the HBsAg produced were cleaved within the MALT cells and presented through the MHC Class I pathway, giving rise to a strong CTL response. On the other hand, only a very small amount of HBsAg generated in the MALT cells are secreted and presented by B cells through the MHC Class II pathway, giving rise to a poor antibody response. This hypothesis is supported by a study which showed that macrophages pulsed with HBsAg were able to elicit strong CTL response when transferred to synergeneic mice. Unfortunately, the authors did not comment on whether such a macrophage transfer would elicit a good antibody response or not. Besides this selective infection of MALT cells, the strong CTL response may have been further affected by the adjuvant, lipopolysaccharide (LPS) of live-attenuated S. typhimurium, that is co-administered with the DNA. Since it has been reported that the LPS of Salmonella can lead to a shift towards Th 1 and CTL immune response, it would not be surprising that when the DNA vaccine is given using live-attenuated S. typhimurium as the carrier, the immune response would be driven markedly towards a strong CTL, but a relatively weak antibody response.

This strongly cellular and relatively absent humoral response may make this vaccine a better candidate as a therapeutic vaccine for chronic HBV carriers than recombinant HBsAg or naked DNA vaccines. Although it was shown recently in a French pilot study that therapy by standard recombinant HBsAg vaccine may be efficient in reducing HBV replication and cancelling the immune tolerance to HBsAg particles in about 50% of people with chronic active HBV replication, this result is probably not applicable to the situation in developing countries because most chronic HBV infections occur as a result of vertical transmission of the virus. This is analogous to the relatively poor response to IFN-_{.}α treatment in our locality, as opposed to a 30-40% response in developed countries. Paradoxically, it was also shown by the same group recently in a HBsAg transgenic mice model for HBV chronic carrier state that the CTL response is most important in the long-term control of transgenic expression of HBsAg in hepatocytes, and the clearance of HBBsAg expression was not associated with cytopathic effect in the liver. There is increasing evidence showing that the CTL response and the associated antiviral cytokines (IFN-_{.}γ, TFN-α_{.}, IL-2) developed are the major determining factors for recovery from HBV infection, and along the same lines, other groups have tried to improve the CTL response through the use of peptide epitomes that are recognized by CTL as immunogen or through lipid modification of antigenic peptides in order to achieve better therapeutic vaccines against HBV. While the humoral response is relatively less important for the clearance of HBC from hepatocytes, its presence may lead to side effects such as serum sickness and immune complex deposition in chronic HBV carriers. Therefore, the uniqueness of this vaccine in generating strong CTL, but minimal humoral response, may make it an effective and safer therapeutic vaccine. In the present study, none of the vaccines showed any toxicity in mice. Further studies in transgenic mice expressing HBsAg could be performed for studying the toxicity induced by the corresponding vaccines during the possible HBsAg clearance.

The strong CTL may make this vaccine a better candidate than recombinant HBsAg vaccine for immunization of donors with subsequent adoptive transfer of their HBV-specific CD8+T cells to HBsAg positive BMT recipients. Clearance of HBV carrier states have been documented in our BMT center that HBsAg positive BMT recipients receiving marrow from HBsAb positive marrow donors. However, this clearance was associated with donors whose HBsAb is a result of natural infections, rather than immunization with the recombinant HBsAg vaccine. Therefore, it would be logical to see whether the present mucosal DNA vaccine, which can elicit a strong CTL response, could lead to clearance of the HBV in the recipients. However, the hepatic flare at the time of immune reconstitution and possible HBV clearance is of major concern if such a strategy is used. Recently, it was shown in our BMT recipients that oral famciclovir 250 mg three times daily, starting at least 1 week prior to BMT and continuing for 24 weeks after transplantation significantly reduced hepatitis due to HBV reactivation in HBsAg positive recipients after allogeneic BMT. Therefore, the approach of simultaneous suppression of HBV replication by famciclovir in BMT recipients and adoptive transfer of HBV-specific CD8+T cells from BMT donors would be a logical approach that is worth trying for the clearance of the HBV in HBsAg positive BMT recipients.

In addition to the potential use as therapeutic vaccine, the present mucosa DNA vaccine is a good candidate for immunizing those people who do not develop an antibody response to the conventional recombinant HBsAg vaccine, as the HBsAg in this vaccine is presented to the immune system in a radically different way. Furthermore, it has been suggested that some apparent non-responders are in fact primed after recombinant HBsAg vaccination, as some can mount an HBsAb response when a dose of recombinant HBsAg is given years later. It was speculated that some of these non-responders may have developed cell-mediated immunity without a humoral response during the primary recombinant HBsAg immunization, and that the humoral response only developed after booster vaccination. This further supports CTL as playing a major role for the prevention of HBV infection.

The non-persistent antibody response associated with oral immunization of live-attenuated Salmonella containing the DNA vaccine may need improvement if such a vaccine is used for global immunization, as it is not clear whether cellular immunity itself is as good as humoral and cellular immunity for the protection against infection. Global immunization against HBV infection requires a vaccine that is highly efficacious, safe, and inexpensive. Recombinant protein vaccines and the recently developed DNA vaccines are generally efficacious. However, since they have to be administered parenterally, transmission of microorganisms due to reuse of needles becomes a mahor problem. Moreover, these vaccines require the production and purification of a large amount of protein or plasmid DNA, and are therefore extremely expensive. Since needle reuse and poverty are major problems in developing countries, where HBV infection is endemic, using either recombinant protein or naked DNA vaccines for global immunization would be far from ideal. Similar results have been obtained when the experiments were performed using live-attenuated S. typhi (Ty21a), a commercially available vaccine that has been shown to be safe, instead of live-attenuated S. typhimurium (data not shown). Therefore, such a mucosal vaccine may be potentially useful for global immunization, as large scale preparation of Ty21a containing the eukaryotically expressible plasmid can be achieved in a relatively inexpensive way.

### Example 6

### Therapeutic Efficacy of Hepatitis B Surface Antigen-Antibodies-Recombinant DNA Composite in HBsAg Transgenic Mice.

Therapeutic efficacy of HBsAg-anti-HBs-recombinant DNA harboring hepatitis B virus (HBV) S gene complex was compared with three other therapeutic vaccine candidates (recombinant HBsAg, HBsAg complexed to anti-HBs antibodies and naked plasmid DNA encoding the HBV S gene). After four injections at 3-week intervals, the most pronounced decrease of serum HBsAg, the highest titer of anti-HBs response, the highest level of interferon-γ produced by splenocytes and potent cytotoxicity T cell response were observed in the HBsAg-anti HBs-sDNA Immunized group. Reduced expression of HBsAg in hepatocytes was also shown. The therapeutic mechanism of HBsAg-anti-HBs-DNA was speculated as modulation of HBsAg presentation via both endogenous and exogenous pathways.

Materials and Methods

Mice

C57BL/6J-TgN (Alb1HBV) 44 Bri mice (H-2^{b}), checked for serum HBsAg positive, anti-HBs negative, and HBsAg positive in the liver and kidney tissues (after being sacrificed), were provided by The Jackson Laboratory (USA). A total of 28 transgenic mice (13 males, and 15 females), 8-12 weeks of age, weight, 16-18 g were used in this study. Normal C57BL/6J mice (H-2b) were bred under standard pathogen-free conditions in the Laboratory Animal Unit of the University of Hong Kong. All mice were housed in cages under standard conditions. The criteria outlined in the ‘Guide for the Care and Use of Laboratory Animals’ (NIH publication 86-23, 1985) were followed.

Immunogens

Recombinant yeast-derived HBsAg (lot YHB 9811223): commercial yeast-derived recombinant hepatitis B vaccine was provided by Beijing Institute of Biological Products (China).

HBsAg-mouse anti-HBs IC: the source of HBsAg used for preparation of IC was from the same lot of vaccine as stated above. The mouse anti-HBs antibodies used were provided by our own laboratory. IC was prepared in excess of HBsAg as described by Qu et al.

Recombinant plasmid DNA with insertion of HBV S gene driven by cytomegalovirus immediate early promoter (s-DNA) was a generous gift from Whalen. Plasmid DNA was amplified and purified by anion exchange column (Qiagen, Hilden, Germany), and finally, resuspended in endotoxin-free sterile physiological saline for injection. All plasmid DNA used were checked for endotoxin (less than 0.25 endotoxin unit/μg) prior to immunization. IC-sDNA was prepared by combining naked plasmid DNA with IC at appropriate ratio.

TABLE 5
Immunogens Used in Different Groups of Transgenic Mice

| | | | Number of animals | | |
|---|---|---|---|---|---|
| Groups | Immunogens | Dose (per mouse) | Male | Female | Total |
| 1 | HBsAg + alum | 2 μg HBsAg | 2 | 4 | 6 |
| 2 | IC^{a} + alum | 2 μg HBsAg | 3 | 3 | 6 |
| 3 | IC-sDNA^{b} | 2 μg HBsAg + | 2 | 3 | 5 |
| | | 100 μg sDNA | | | |
| 4 | s-DNA | 100 μg sDNA | 3 | 2 | 5 |
| 5 | Unimmunized | NA^{c} | 3 | 3 | 6 |
| Total | | | 13 | 15 | 28 |

^{a}IC - HBsAg-anti-HBs complex.
^{b}sDNA - recombinant plasmid DNA harboring S gene.
^{c}NA - non-applicable.

Immunization

Twenty-eight HBsAg transgenic mice were numbered and randomly divided into five groups and immunized with different immunogens (Table 5). To exclude the effect of anesthesia over the immune response in mice, all immunized mice were anesthetized with identical dose of sodium barbital, and all immunogens were injected into the tibialis anterior muscle of both hind legs of mice. The immunization was given in four doses every 3 weeks over 12 weeks, and on week 14, mice were boosted with the same immunogen 7 days prior to sacrificing the mice for cell-mediated immune response assay.

Determination of Immune Responses

Serum samples were taken before each dose of immunization for the determination of HBsAg and anti-HBs. Both serum HBsAg and anti-HBs were assayed by ELISA (BIOKIT, S.A. Spain). For HBsAg quantification a panel of HBsAg calibrators (Abbott Diagnostics, Chicago) was applied in the assay. The level of anti-HBs was quantified using standard positive controls (10-100 mIU/ml) provided with the kits. The animals were sacrificed on week 15. The spleen cells from all animals were assayed for HBsAg specific Th 1 and Th 2 cell cytokines 5×10⁵ splenocytes from each mouse were cultured in 10% calf serum-RPMI 1640, stimulated with 10 μg/ml of recombinant HBsAg at 37° C. for 3 days, and supernatants of cultured cells were collected and interferon-γ and interleukin-4 were assayed by ELISA using OptEIA kits (Phar-Mingen, USA).

Cells were further cultured by adding 25 IU/ml of murine recombinant IL-2 (R&D Systems, USA) for additional 4-5 days to expand specific T cells. The cytotoxicity T cell (CTL) activity of the splenocytes was measured in triplicate using a standard 4 h calcein release assay in U-bottom 96-well microplates. Target cells used in CTL assays were the splenocytes of normal C57/6J, infected either with 10 PFU/cell of a recombinant vaccinia virus which harbored the HBsAg gene (vaccinia-HBsAg virus, abbreviated as Vac-HBsAg) or with vaccinia virus (Vac, negative control) for 12 h. Target cells were labeled immediately before use by incubating cells in 2 □M calcein AM (molecular Probes Inc., USA) for 40 min at 37° C. The expanded effector spleen cells were purified and resuspended in 10% calf serum-RPMI 1640, mixed with 5000 calcein AM labeled targets, at effector/target (E:T) ratios of 100/0.3. The plates were centrifuged at 100×g for 3 min and further incubated at 37° C. for 4 h. The cytolysis of the targets was determined by measuring the fluorescence intensity (FI). The percentages of specific cytolysis were calculated as follows:

$$( 1 - \frac{Experimental FI - Total lysis FI}{Target control FI - Total lysis FI} ) \times 100 \%$$

Immunohistopathological Study

After sacrificing the mice, liver and kidney tissues were either snap frozen in liquid nitrogen or fixed in 10% of buffered formaldehyde, followed by embedding in paraffin. Sections were examined by immunohistochemical staining for HBsAg expression using HBsAg detection kits (Dako, USA) or by haemotoxylin and eosin staining for studying histopathological changes. Tissue sections were read under code by pathologists from two independent laboratories.

Statistical Analysis

The significance of differences between groups was analyzed by paired Student's t-test.

Results

Serum HBsAg Levels

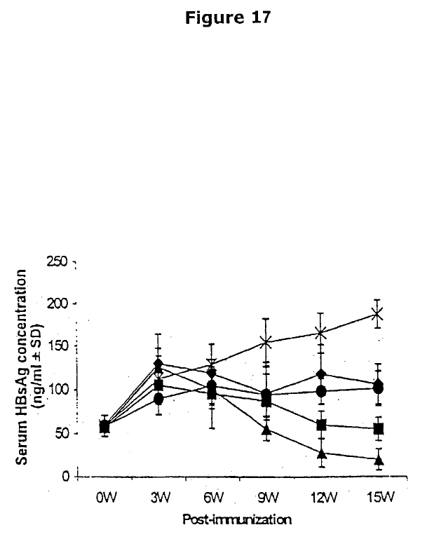

The results are summarized in FIG. 17. The serum HBsAg levels in samples obtained on weeks 0 and 3 were essentially the same for all five groups of animals. In the control unimmunized groups, serum antigen level increased over the 15-weeks period of observation from the mean value of 113±13 to 189±17 ng/ml on week 15 (P<0.02). In contrast to the controls, the increase of the antigen level was arrested in all immunized groups. In the IC immunized group, compared to the antigen level on week 3, decline in the antigen level was first evidenced on week 12 (P<0.05) and the antigen sustained at the similar level up to week 15. Immunization with IC-sDNA induced the most marked and rapid decrease in the serum antigen levels. The decline in antigen levels was first evidenced in this group of mice on week 9. Serum HBsAg level was 126±22 ng/ml on week 3 in this group, but declined to 56±14 ng/ml (P<0.02) on week 9. The decline continued over the subsequent 6 weeks, reaching a low mean level of 28 ng/ml on week 12 and serum HBsAg was sustained at the similarly low level until termination of the experiment on week 15.

Anti-HBs Antibodies

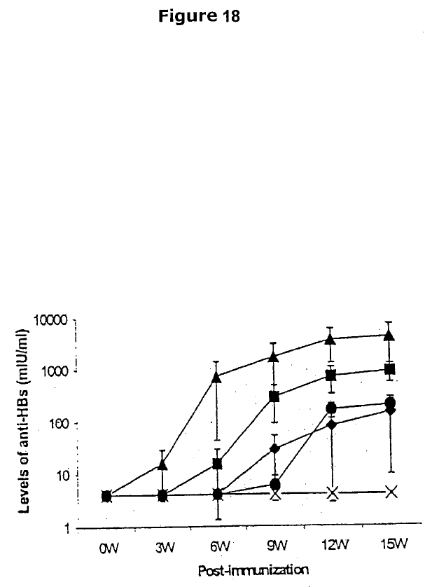

Immunication with IC-sDNA complex elicited the most vigorous antibody response with anti-HBs appearing 3 weeks after the first dose of vaccine (FIG. 18). The antibody rose rapidly after the receipt of the second dose and increased continuously which reached 4223±3301 mIU/ml on week 15. In IC and HBsAg immunized groups, antibody response was less vigorous, and the levels were 904±359 and 149±149 mIU/ml, respectively. Antibody response induced by DNA immunization was similar to that elicited by HBsAg alone (203±59 mIU/ml). None of the unimmunized control animals produced detectable level of anti-HBs throughout the course of the experiment.

| | Number | IFN-γ (pg/ml | | |
|---|---|---|---|---|
| Immunogens | of Mice | 24 h | 48 h | 72 h |
| HBsAg | 6 | 98^{a} (106)^{b} | 303 (211) | 607 (502) |
| IC | 6 | 234 (124) | 1044 (688) | 3396 (3180) |
| IC-sDNA | 5 | 679 (683) | 2980 (2280) | 13 396 (16 881) |
| sDNA | 5 | 132 (52) | 815 (573) | 2044 (639) |
| Unimmunuzed | 6 | 33 (55) | 100 (106) | 75 (93) |

^{a}Average of interferon-γ
^{b}S.D. of interferon-γ

Cytokine Production

Interferon_{.}-production from HBsAg-stimulated spleen cells of each immunized group is shown in Table 6. Interferon-_{.}γ level varied broadly from mouse to mouse in each group. IC-sDNA elicited a vigorous Th 1-type immune response, as shown by the production of the highest level of interferon-γ_{.}, and IL-4 production was slightly increased in the IC-sDNA group, which was not of statistical significance (data not shown).

Cytolytic T Cell Response

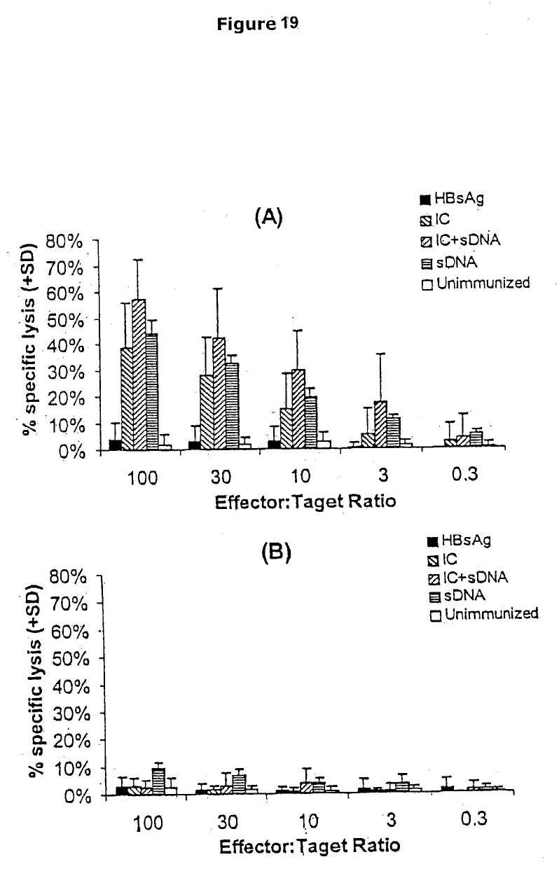

The result of HBV specific CTL activity in all immunized groups are shown in FIG. 19. In IC, DNA, IC-sDNA immunized groups, mouse spleen cells were cytotoxic against Vac-HBsAg recombinant virus infected target cells, but they did not exhibit cytotoxicity against the control vaccinia virus infected cells. CTL response was barely induced in HBsAg immunized mice.

Histology and Expression of HBsAg in Liver

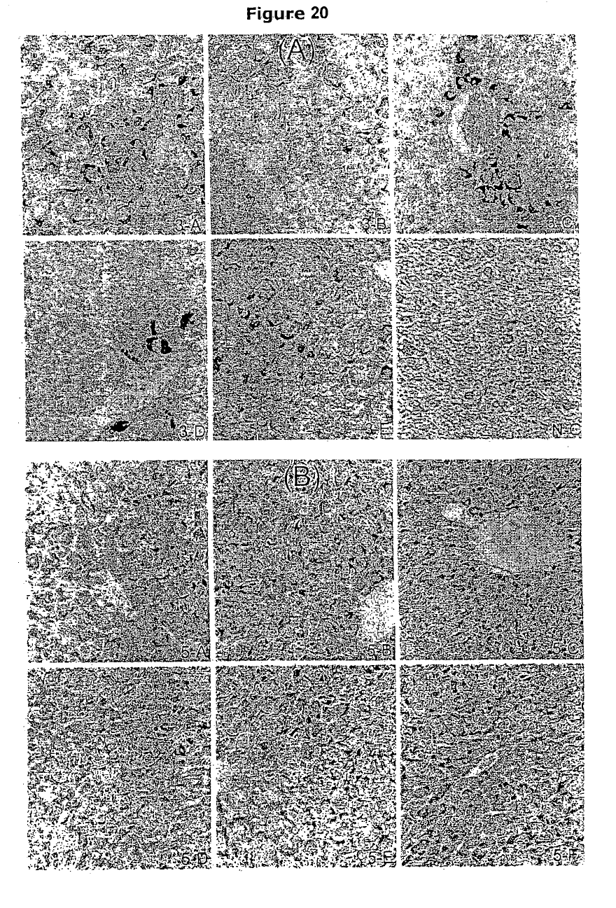

No histopathological changes in the liver or kidney from all groups of animals were observed. By immunohistochemical staining, except for the mice immunized with IC-sDNA, expression of HBsAg in liver tissues from the animals was similar to that in the control group. Fewer HBsAg positive hepatocytes were found in liver sections from IC-sDNA immunized mice. The extent of reduced expression of HBsAg varied among mice in this group, and was most pronounced in two mice (FIG. 20).

Discussion

In a pilot study, we have previously shown that HBsAg complexed to human HBIG (IC) was effective in reducing or clearance of serum HBV viremia in chronic hepatitis B patients. However, no decrease in the serum HBsAg level of the treated patients was observed. When the immunotherapeutic mechanism of this antigen-antibody complex was studied in normal Balb/c mice, we discovered that when plasmid DNA was added to the antigen-antibody complex to generate a new composite, more potent humoral and cellular immune response could be induced. However, when vector plasmid DNA was added to HBsAg-anti-HBs complex to immunize mice, only enhanced anti-HBs response was observed; whereas when recombinant plasmid DNA harboring HBsAg gene was added to the complex, both humoral and cell-mediated immune response were enhanced. These results suggested that HBsAg-anti-HBs-DNA complex can be used as a new approach to treat HBV carriage and the chronic disease associated with it. To test this possibility and to compare the efficacy of this composite with other described immunotherapeutic vaccine candidates, we used four immunogens to immunize the same lineage of transgenic mice. The immunization schedule, route and volume of inoculation, anesthetization of animals and genders of mice distributed in each immunized group were designed to minimize bias in results obtained.

In the lineage of HBV-transgenic mice used in this study, HBsAg was expressed by virtually all the hepatocytes and the antigen was detected in increasing concentrations in the consecutive serum samples taken over the 15-weeks duration of our experiment. Presumably, this may be because the rate of antigen production exceeded the rate of disposal, such that there is a tendency for the antigen to accumulate as the animal aged. Though we did not succeed to clear the serum HBsAg nor eliminate HBsAg expression in hepatocytes, the pronounced reduction of HBsAg expression in IC-sDNA immunized mice was encouraging.

In this study, even the protein vaccine was able to break the immune tolerance and induced a weak HBV specific immune response in these animals. The immune response induced by s-DNA immunization in this study was not as pronounced as that reported by others, which could be due to a different construction of the recombinant plasmid or due to different mouse strain used. However, naked DNA immunization did induce CTL response, production of interferon-γ_{.} and anti-HBs, which were adequate to arrest the increase of serum antigen level in animals. The IC immunogen induced an effective but moderate immune response, which was shown by good CTL response, high interferon-γ_{.} production, anti-HBs response and a decline in serum antigen level. IC-sDNA immunization resulted in the best effective response, by marked decrease of serum HBsAg, inducing high level of interferon-γ_{.}, high titer of anti-HBs and effective CTL activity. However, in most of the animals, the decrease in serum HBsAg level was not well correlated with the expression of HBsAg in liver tissues. This discrepancy strongly suggest that the decrease in serum HBsAg was mainly due to the neutralizing effect of induced anti-HBs, which was not effective in clearing the HBsAg in hepatocytes.

Only in sections of the liver tissues from IC-sDNA immunized mice, fewer HBsAg positive cells were found. In chimpanzees, a noncytopathogenic antiviral mechanism was described and cytokines played important roles. Due to technical problems, we did not succeed in assaying the HBsAg mRNA in these liver tissues. However, since the level of interferon-γ_{.} induced in splenocytes was the highest in this group of mice, the down-regulation of HBsAg expression could possibly be mediated via cytokines, e.g., interferon-γ_{.}.

The in vitro cytolytic activity was not observed in liver tissue sections, which could be due to lack of effector cells in the liver tissue of transgenic mice. By haemotoxylin eosin staining, very few mononuclear cells were found in the liver tissues of immunized and control transgenic mice. In addition, the target cells were different between in vitro and in vivo. The hepatocytes expressing the transgene (HBsAg) as targets in vivo could react differently from the recombinant Vac-HBsAg virus infected splenocytes in vitro.

We have shown that by IC immunization, enhanced uptake of HBsAg via the Fc receptors on macrophages and dendritic cells occurred and potentiated in vitro specific lymphocyte proliferation, possibly through the modulated presentation of HBsAg by professional antigen presenting cells. We speculated that when IC-DNA composite was used for intramuscular injection, the professional APCs drawn by IC to the site of inoculation would provide an excellent micro-environment for naked DNA to contact and interact with APC, and presumably, when IC and DNA were co-ingested and processed, the combination of both exogenous and endogenous pathways of antigen presentation could induce potent host immunce responses. In addition, the naked DNA in this composite could be protected from enzyme-mediated degradation and be stabilized, and the CpGs in plasmid DNA could serve as the adjuvant to enhance the immunogenicity of the complex. More studies on the immune mechanisms of this composite will elucidate the synergistic therapeutic effects in the trangenic mice model. Since different lineage of mice used and different constructs of immunogens employed could influence the outcome of immunotherapeutic studies, IC-sDNA immunization should be studied in other transgenic mice models, especially in those with active virus replication.

### REFERENCES

1. World Health Organization. 1998. The World Health Report. Geneva:WHO.

2. Lok A. S. F., C. L. Lai, P. C. Wu, E. K. Leung. 1998. Long-tern follow-up in a randomised controlled trial of recombinant alpha 2-interferon in Chinese patients with chronic hepatitis B infection. Lancet 2:298.

3. Tassopoulos N. C., R. Volpes, G. Pastore, J. Heathcote, M. Buti, R. D. Goldin, S. Hawley, J. Barber, L. Condreay, D. F. Gray. 1999. Efficacy of lamivudine in patients with hepatitis B e antigen-negative/hepatitis B virus DNA-positive (precore mutant) chronic hepatitis B. Lamivudine Precore Mutant Study Group. Hepatology 29:889.

4. Lau D. T., E. Doo, Y. Park, D. E. Kleiner, P. Schmid, M. C. Kuhns, J. H. Hoofnagle. 1999. Lamivudine for chronic delta hepatitis. Hepatology 30:546.

5. Jardi R., M. Buti, F. Rodriguez-Frias, M. Cotrina, X. Costa, C. Pascual, R. Esteban, J. Guardia. 1999. Rapid detection of lamivudine-resistant hepatitis B virus polymerase gene variants. J. Virol. Methods 83:181.

6. Rehermann B., P. Fowler, J. Sidney, J. Person, A. Redeker, M. Brown, B. Moss, A. Sette, F. V.Chisari. 1995. The cytotoxic T lymphocyte response to multiple hepatitis B virus polymerase epitopes during and after acute viral hepatitis. J. Exp. Med. 181:1047.

7. Chisari F. V. 1997. Perspectives series: Host/pathogen interactions. J. Clin. Invest. 99:1472.

8. Lau G. K., R. Liang, C. K. Lee, S. T. Yuen, J. Hou, W. L. Lim, R. Williams. 1998. Clearance of persistent hepatitis B virus infection in Chinese bone marrow transplant recipients whose donors were anti-hepatitis B core- and anti-hepatitis B surface antibody-positive. J. Infect. Dis. 178:1585.

9. Akbar F and Onji M. 1998. Hepatitis B virus (HBV)-transgenic mice as an investigative tool to study immunopathology during HBV infection. Int. J. Exp. Path. 79:279-291.

10. Wirth, S., L. G. Guidotti, K. -I. Ando, H. J. Schlicht, F. V. Chisari. 1995. Breaking tolerance leads to autoantibody production but not autoimmune liver disease in hepatitis B virus envelope transgenic mice. J. Immunol. 154:2504.

11. Shimizu Y., L. G. Guidotti, P. Fowler, F. V. Chisari. 1998. Dendritic cell immunization breaks cytotoxic T lymphocyte tolerance in hepatitis B virus transgenic mice. J. Immunol. 161:4520.

12. Wen Y M., Xiong S D, Zhang W. 1994. Solid matrix-antibody-antigen complex can clear viremia and antigenemia in persistent duck hepatitis B virus infection. J. Gen. Virol. 75:335-339.

13. Zheng B J, Ng M H, He L F, Yao X, Chan K W, Yuen K Y, Wen Y M. 2001. Therapeutic efficacy of hepatitis B surface antigen-antibody-recombinant composite in HBsAg transgenic mice. Vaccine 19:4219-4225.

14. Zheng B J, Tsoi H W, Woo P C Y, Ng M H, Yuen K Y. A crucial role of macrophage in the immune responses to oral vaccination against hepatitis B virus in a murine model. Vaccine. 2001; 19:4219-4225.

15. Shimizu Y., L. G. Guidotti, P. Fowler, F. V. Chisari. 1998. Dendritic cell immunization breaks cytotoxic T lymphocyte tolerance in hepatitis B virus transgenic mice. J. Immunol. 161:4520.

16. Schirmbeck R., J. Wild, D. Stober, H. E. Blum, F. V. Chisari, M. Geissler, J. Reimann. 2000. Ongoing murine T1 or T2 immune responses to the hepatitis B surface antigen are excluded from the liver that expresses transgene-encoded hepatitis B surface antigen. J. Immunol 164:4235.

17. Mancini M, Hadchouel M, Davis H L, Whalen R G, Tiollais P, Michel M L. 1996. DNA-mediated immunization in a transgenic mouse model of the hepatitis B surface antigen chronic carrier state. Proc. natl. Acad. Sci. USA 93:12496-12501.

18. Zheng B J, Ng M H, Chan K W, Tam S, Woo P C Y, Ng S P, Yuen K Y. A Single Dose of Oral DNA Immunization Delivered by Attenuated Salmonella typhimurium Down-regulates Transgene Expression in HBsAg Transgenic Mice (Submitted to J. Immunol.)

19. Hoiseth S K, Stocker B A. 1981. Aromatic-dependent Salmonella typhimurium are non-virulent and effective as live vaccines. Nature 291:238-239.

20. Darji A., C. A. Guzman, B. Gerstel, P. Wachholz, K. N. Timmis, J. Wehland, T. Chakraborty, S. Weiss. 1997. Oral somatic transgene vaccination using attenuated S. typhimurium. Cell. 91:765.

21. Chisari, F. V., K. Klopchin, T. Moriyama, C. Pasquinelli, H. A. Dunsdorf, S. Sell, C. A. Pinkert, R. L. Brinster, R. D. Palmiter. 1989. Molecular pathogenesis of hepatocellular carcinoma in hepatitis B virus transgenic mice. Cell 59:1145.

Ausubel, F. M. et al., Current Protocols in Molecular Biology, John Wiley & Sons, New York, N.Y., 1989.

Harlow and Lane, Antibodies: A Laboratory Manual, Cold Spring Harbor Lab., New York, 1988.

Sambrook et al., Molecular Cloning: A Laboratory Manual, 2^{nd} Ed., Cold Spring Harbor Laboratory Press, Plainview, N.Y., 1989.

Zinkernagel, R. M. Fundamental Immunology, 3rd edition. Raven Press, Paul, W.-editor. Chapter 34, pp. 1211-1250, 1993.

We claim:

1. An oral DNA composition for improving an impaired immunity associated with chronic infection of HBV and for suppressing transgene expression for a protracted period of time comprising:
an attenuated strain of bacteria which preferentially targets phagocytic cells, wherein cells of the bacterial strain are transformed by a plasmid vector comprising:
one or more genes, or complementary DNA thereof, coding for at least a portion of a hepatitis B viral protein or peptide or antigenic portion thereof;
a promoter operably linked to the gene or complementary DNA permitting expression thereof in an eukaryotic environment; and
an auxotrophic mutation which causes the cells of the bacterial strain to undergo autolysis once they have gained entry into the phagocytic cells; and
a pharmaceutically acceptable carrier.

2. An oral DNA composition according to claim 1 wherein the phagocytic cells are those of the intestinal mucosa.

3. An oral DNA composition according to claim 1 wherein the phagocytic cells include inflammatory cells recruited in response to an infection.

4. An oral DNA composition according to claim 1 wherein the attenuated strain of bacteria is Salmonella typhimurium aroA.

5. An oral DNA composition according to claim 1 wherein the attenuated strain of bacteria is selected from the group consisting of attenuated strain of Salmonella typhimurium strain S7207 and attenuated strain of Salmonella typhi Strain Ty21a.

6. A process for inducing a cell-mediated immune response in a chronically infected HBV carrier comprising:
orally administering to the HBV carrier an effective amount of an attenuated bacterial strain which preferentially targets phagocytic cells, wherein cells of the bacterial strain undergo autolysis when taken up by the phagocytic cells, thereby causing release of a plasmid vector contained therein which is capable of expressing at least a portion of a HBV genome in an eukaryotic environment; and
inducing a cell-mediated immune response in the HBV carrier and suppressing HBV expression.

7. A process according to claim 6 wherein the phagocytic cells are those of the intestinal mucosa.

8. A process according to claim 6 wherein the phagocytic cells include inflammatory cells recruited in response to an infection.

9. A process according to claim 6 wherein the attenuated strain of bacteria is Salmonella typhimurium aroA.

10. A process according to claim 6 wherein the attenuated strain of bacteria is selected from the group consisting of attenuated strain of Salmonella typhimurium strain S7207 and attenuated strain of Salmonella typhi strain Ty21a.

11. A process according to claim 6 wherein the plasmid vector comprises: one or more genes, or complementary DNA thereof, coding for at least a portion of a hepatitis B viral protein or peptide; a promoter operably linked to the hepatitis B gene or complementary DNA which allows expression thereof in an eukaryotic environment; and an auxotrophic mutation that causes the bacteria to undergo autolysis upon entry into the phagocytic cells.
